(19) United States
(12) Patent Application Publication
(10) Pub. No.: US 2021/0172929 A1
(43) Pub. Date: Jun. 10, 2021

(54) Multi-Pore Device with Material Sorting Applications

(71) Applicant: Nooma Bio, Inc., Santa Cruz, CA (US)

(72) Inventors: David Alexander, Santa Cruz, CA (US); William B. Dunbar, Santa Cruz, CA (US)

(21) Appl. No.: 17/112,846

(22) Filed: Dec. 4, 2020

Related U.S. Application Data
(60) Provisional application No. 62/944,271, filed on Dec. 5, 2019.
(60) Provisional application No. 62/962,509, filed on Jan. 17, 2020.

(51) Int. Cl. G01N 33/487; C12Q 1/6874; G01N 27/447

(52) U.S. Cl. CPC G01N 33/48721; B82Y 5/00; G01N 27/44791; C12Q 1/6874

(57) ABSTRACT

Multi-pore devices and method for material sorting are described. A multi-pore device can include first channel coupled to a first nanopore and a second channel coupled to a second nanopore. The device can also include sensing circuitry for measuring electrical signals associated with a target at a respective nanopore, and control circuitry for controlling motion of the target at a respective nanopore. The device can include and/or switch between sensing and control modes for each of the first nanopore and the second nanopore. The device(s) can implement methods for generating and detecting signals upon translocation of target material and non-target material into a respective nanopore, and based upon signatures derived from the signals, sort the target material or non-target material for various downstream applications.

### CROSS REFERENCE TO RELATED APPLICATIONS

This application claims priority to U.S. Provisional Application No. 62/944,271 filed on Dec. 5, 2019 and U.S. Provisional Application No. 62/962,509 filed on Jan. 17, 2020. The content of each of the above referenced applications is incorporated by reference in its entirety.

### BACKGROUND

A nanopore is a nano-scale conduit that forms naturally as a protein channel in a lipid membrane (a biological pore), or is engineered by drilling or etching the opening in a solid-state substrate (a solid-state pore). When such a nanopore is incorporated into a nanodevice comprising chambers that are separated by the nanopore, a sensing device can be used to apply a trans-membrane voltage and measure current through the pore.

Nanopores offer great promise for inexpensive target material detection and sequencing application. Some obstacles to nanopore sequencing, however, include: (1) the lack of sensitivity sufficient to accurately determine the identity of each nucleotide in a nucleic acid for de novo sequencing (the lack of single-nucleotide sensitivity), (2) the ability to regulate and control the delivery rate of each nucleotide unit through the nanopore during sensing, and (3) the ability to selectively retrieve and/or further process target material from non-target material of a sample upon sensing and discriminating target material from non-target material. Enrichment of target nucleic acids without requiring PCR remains a challenge for most single-molecule techniques, including long-read sequencing methods and mapping methods with nanopores or with optical imaging of molecules immobilized or confined in nanochannels. Furthermore, when PCR is required, enriching for target amplicons from background can still be a challenge, e.g., for cell-free DNA analysis. Thus, there is a need for a single-molecule approach to serially detecting and then fluidically sorting molecules, to segregate target molecules from non-target molecules, that can work upstream of PCR or non-PCR workflows.

### SUMMARY

Embodiments relate to a multi-pore nanopore device and methods of sorting target material from non-target material using embodiments of the nanopore device.

In embodiments, a multi-pore nanopore device can include first channel coupled to a first nanopore and a second channel coupled to a second nanopore, where material can be translocated from the first nanopore to the second nanopore and/or another region of the multi-pore device. The device can also include sensing circuitry for measuring electrical signals associated with a target at a respective nanopore, and control circuitry for controlling motion of the target at a respective nanopore. The device can include and/or switch between sensing and control modes for each of the first nanopore and the second nanopore. The device(s) can implement methods for generating and detecting signals upon translocation of target material and non-target material into a respective nanopore, and based upon signatures derived from the signals, sort the target material or non-target material for various downstream applications.

In embodiments, a method implemented by way of the multi-pore nanopore device can include: receiving a sample, having one or more target polynucleotides, at a first channel of a nanopore device; translocating the polynucleotides into a first nanopore coupled to the first channel, upon application of a control voltage across the first nanopore by a control circuit of the first nanopore; generating a signal in coordination with translocation of each polynucleotide into the first nanopore and applying a sensing voltage across the first nanopore by a sensing circuit of the first nanopore; detecting a signature of each translocated polynucleotide, the signature derived from the signal; and based upon the signature, translocating the polynucleotide into a second portion of the multi-pore nanopore device. Aspects of portions of the devices into which target or non-target material can be translocated and/or from which target or non-target material can be retrieved are further described below.

According to various applications, the invention(s) described can include methods for detecting and sorting long read sequences of polynucleotides, with downstream amplification (e.g., using polymerase chain reaction (PCR) operations). Additionally or alternatively, the invention(s) can include systems and methods for detection and sorting of barcoded material (e.g., variants of material associated with antibiotic resistance, variants of material associated with drug resistance). Additionally or alternatively, the invention(s) can include systems and methods for sorting vectors (e.g., lentiviral vectors, whole phages, etc.), proteins (e.g., antibodies associated with SARS-CoV-2, other antibodies, other proteins), nucleic acid origami libraries, previously unidentified molecules that can be used as sorting agents, and/or other target material. Additionally or alternatively, the invention(s) can include systems and methods for enriching target material (e.g., bacteria from whole blood), capturing plasmids, sorting populations (e.g., sorting wild-type vs. non-wild-type genetic material), and/or other downstream applications of material sorting.

In variations, sorting can be performed iteratively and/or multiple times, such that target material can be enriched from a sample.

In embodiments, the invention(s) enable enrichment of target amplicons from background (e.g., for cell-free DNA analysis), with a single-molecule approach. The approach provides systems and methods for serially detecting and then fluidically sorting molecules, to segregate target molecules from non-target molecules, that can work upstream of PCR or non-PCR workflows. Discussed approaches could also segregate other types of target analytes, including chromosomal fragments comprising histones that are detected as having a target modification, from those fragments with histones that do not have the modification, and sorting facilitating enriching for the modified histone containing chromosomal fragment for subsequent epigenetic analysis, such as ChIP-seq or ATAC-seq or bisulfate sequencing.

Additional embodiments and variations of the invention(s) are further described below.

### BRIEF DESCRIPTION OF DRAWINGS

The disclosed embodiments have advantages and features that will be more readily apparent from the detailed description, the appended claims, and the accompanying figures (or drawings). A brief introduction of the figures is below.

FIG. 1 depicts an embodiment of a nanopore device for material sorting, in accordance with one or more embodiments.

FIG. 2 depicts an example nanopore device with two nanopores, in accordance with one embodiment.

FIG. 3A depicts example circuitry incorporating the two nanopores of an example nanopore device, in accordance with one embodiment.

FIG. 3B depicts example circuitry incorporating the two nanopores of an example nanopore device, in accordance with one embodiment.

FIG. 4 depicts an example two nanopore device with a sensing circuitry and a control circuitry option for each pore, and a switch between the two options for each pore, in accordance with one embodiment.

FIG. 5A depicts an example two nanopore device in a first configuration, in accordance with one embodiment.

FIG. 5B depicts an example two nanopore device in a second configuration, in accordance with one embodiment.

FIG. 6 depicts a flow process for sequencing a molecule such as a polynucleotide, in accordance with an embodiment.

FIG. 7 depicts a flow processing for sorting target material from non-target material of a sample, in accordance with an embodiment.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

In some instances, a component (e.g., a nucleic acid component; a protein component; and the like) includes a label moiety. The terms “label”, “detectable label”, or “label moiety” as used herein refer to any moiety that provides for signal detection and may vary widely depending on the particular nature of the assay. Label moieties of interest include both directly detectable labels (direct labels)(e.g., a fluorescent label) and indirectly detectable labels (indirect labels)(e.g., a binding pair member). A fluorescent label can be any fluorescent label (e.g., a fluorescent dye (e.g., fluorescein, Texas red, rhodamine, ALEXAFLUOR® labels, and the like), a fluorescent protein (e.g., green fluorescent protein (GFP), enhanced GFP (EGFP), yellowfluorescent protein (YFP), red fluorescent protein (RFP), cyan fluorescent protein (CFP), cherry, tomato, tangerine, and any fluorescent derivative thereof), etc.). Suitable detectable (directly or indirectly) label moieties may include any moiety that is detectable by spectroscopic, photochemical, biochemical, immunochemical, electrical, optical, chemical, or other means. For example, suitable indirect labels include biotin (a binding pair member), which can be bound by streptavidin (which can itself be directly or indirectly labeled). Labels can also include: a radiolabel (a direct label)(e.g., ³H, ¹²⁵I, ³⁵S, ¹⁴C, or ³²P); an enzyme (an indirect label)(e.g., peroxidase, alkaline phosphatase, galactosidase, luciferase, glucose oxidase, and the like); a fluorescent protein (a direct label)(e.g., green fluorescent protein, red fluorescent protein, yellow fluorescent protein, and any convenient derivatives thereof); a metal label (a direct label); a colorimetric label; a binding pair member; and the like. By “partner of a binding pair” or “binding pair member” is meant one of a first and a second moiety, wherein the first and the second moiety have a specific binding affinity for each other. Suitable binding pairs include, but are not limited to: antigen/antibodies (for example, digoxigenin/anti-digoxigenin, dinitrophenyl (DNP)/anti-DNP, dansyl-X-anti-dansyl, fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Any binding pair member can be suitable for use as an indirectly detectable label moiety.

Any given component, or combination of components can be unlabeled, or can be detectably labeled with a label moiety. In some cases, when two or more components are labeled, they can be labeled with label moieties that are distinguishable from one another.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a ribonucleoprotein complex” includes a plurality of such complexes and reference to “the mutant dystrophin gene” includes reference to one or more mutant dystrophin genes and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### DETAILED DESCRIPTION

### Nanopore Devices

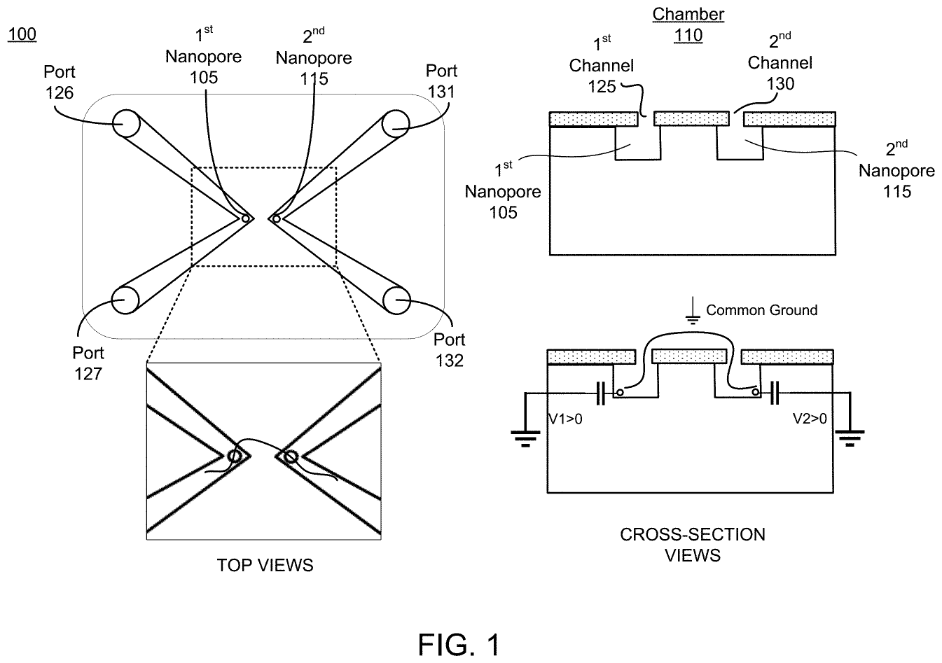

In some embodiments, a dual-pore nanopore device includes at least one nanopore (as shown in FIG. 1) that forms an opening in a structure separating an interior space of the nanopore device into two volumes. As shown in FIG. 1, the device 100 includes a first nanopore 105 in fluid communication with a first channel 125 and a second nanopore 115 in fluid with a second channel 130, where the device 100 includes a common chamber 110 in fluid communication with both the first channel 125 and the second channel 130. As shown in FIG. 1, each of the first channel 125 and the second channel 130 includes channel ports (e.g., ports 126 and 131, ports 127 and 132) into which or out of which polynucleotides of a sample can be delivered, where circuitry (described in further detail below) provides driving and sensing functions of the device 100. In particular, as shown in FIG. 1 (bottom left, bottom right), the device 100 can process polynucleotides (e.g., polynucleotide 10) and/or other molecules of a sample by translocating the polynucleotides and/or other molecules between the first nanopore 105 and the second nanopore 115, between the first nanopore 105 and the common chamber 110, and/or between the second nanopore 115 and the common chamber 110.

The nanopore devices also includes at least a sensor in electrical communication with the opening and configured to identify objects (for example, by detecting changes in electrical signal parameters indicative of objects) passing through the nanopore. Nanopore devices that may be used for the methods and systems described herein are also disclosed in PCT Publication Nos. WO/2013/012881 and WO/2018/236673, U.S. Application Publication No. 2017/0145481, U.S. Pat. Nos. 9,863,912, and 10,488,394, which are hereby incorporated by reference in their entirety. Amplifiers and circuitry in the nanopore devices that may be used for the methods and systems are also disclosed in U.S. Application Publication No. 2017/0145481, which is hereby incorporated by reference in its entirety.

In some embodiments, the nanopore(s) in the nanopore device(s) are nanoscale or microscale in relation to characteristic feature dimensions. In one aspect, each pore has a size that allows a small or large molecule (e.g., nucleic acid molecule or fragment) or microorganism to pass. In examples, nanopores can have a diameter from 1 nm through 100 nm; however, in variations of the examples, nanopores can have a diameter less than 1 nm or greater than 100 nm. In some embodiments, the diameter of the pores range from about 2 nm to about 50 nm. In some embodiments, the diameter of the pores is about 20 nm. In variations, a nanopore has a depth ranging from 1-10,000 nm; however, in other variations, a nanopore can have a depth less than 1 nm or greater than 10,000 nm. Furthermore, during an experimental run, nanopore dimensions may vary (within a suitable range), as described in further detail below.

In some embodiments, each of the pores in the dual-pore device independently has a depth. In one embodiments, each pore has a depth that is least about 0.3 nm. In some embodiments, each pore has a depth that is at least about 0.6 nm, 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, or 90 nm. In some embodiments, each pore has a depth that is no more than about 100 nm. Alternatively, the depth is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 or 10 nm. In some embodiments, the pore has a depth that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm. In some embodiments, the first pore has a depth of at least about 0.3 nm separating the first fluidic channel and the chamber and the second pore has a depth of at least about 0.3 nm separating the chamber and the second fluidic channel.

In some aspects, each of the pores in the dual-pore device independently has a size that allows a small or large molecule or microorganism to pass. In some embodiments, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 35 nm, 40 nm, 45 nm, 50 nm, 60 nm, 70 nm, 80 nm, 90 nm or 100 nm in diameter.

In some aspects, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some embodiments, a nanopore of a nanopore device has a substantially round shape. “Substantially round”, as used here, refers to a shape that is at least about 80 or 90% in the form of a cylinder. However, in alternative embodiments, a nanopore device can include nanopores that are square, rectangular, triangular, oval, hexangular, or of another morphology.

In some embodiments, the nanopore extends through a membrane. For example, the pore may be a protein channel inserted in a lipid bilayer membrane or it may be engineered by drilling, etching, or otherwise forming the pore through a solid-state substrate such as silicon dioxide, silicon nitride, grapheme, or layers formed of combinations of these or other materials.

In some embodiments, nanopores of a device can be spaced apart at distances ranging from 5-15,000 nm. In some embodiments, the nanopores of a device can be spaced apart at distances ranging from 10 to 1000 nm. However, in other variations, nanopores can be spaced apart less than 5 nm or greater than 15,000 nm. Furthermore, nanopores can be arranged in any position so long as they allow fluid communication between the chambers and have the prescribed size and distance between them. In some embodiments, the first pore and the second pore are about 10 nm to 500 nm apart from each other. In some embodiments, the first pore and the second pore are about 500 nm apart from each other. In one variation, the nanopores are placed so that there is no direct blockage between them. Still, in one aspect, the pores are substantially coaxial.

In some cases, the diameter of the pores ranges from about 2 nm to about 50 nm. In some cases, the diameter of the pore is about 20 nm. In some cases, the diameter of the first and/or second pore ranges from about 2 nm to about 50 nm. In some cases, the diameter of the first and/or second pore ranges from about 2 nm to about 8 nm. In some cases, the diameter of the first and/or second pore ranges from about 10 nm to about 20 nm. In some cases, the diameter of the pore ranges from about 20 nm to about 30 nm. In some cases, the diameter of the first and/or second pore ranges from about 30 nm to about 40 nm. In some cases, the diameter of the first and/or second pore ranges from about 40 nm to about 50 nm. In some cases, the diameter of the first and/or second pore is about 2 nm, about 4 nm, about 6 nm, about 8 nm, about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm. In some cases, the diameter of the first and/or second pore is about 19 nm. In some cases, the first pore and the second pore have the same diameters. In some cases, the diameter of the first and/or second pore is about 21 nm. In some cases, the diameter of the first and/or second pore is about 22 nm. In some cases, the diameter of the first and/or second pore is about 23 nm. In some cases, the diameter of the first and/or second pore is about 24 nm. In some cases, the diameter of the first and/or second pore is about 25 nm. In some cases, the diameter of the first and/or second pore is about 27 nm. In some cases, the diameter of the first and/or second pore is about 29 nm. In some cases, the first pore and the second pore have different diameters. In some cases, the diameter of the pore is about 20 nm.

In some embodiments, the device comprises a geometrically constrained fluidic volume. In some cases, the geometrically constrained fluidic volume is a fluidic channel. In some cases, the device comprises a first fluidic channel. As used herein, the term “upper chamber” is used interchangeably with the term “fluidic channel” and “geometrically constrained fluidic volume”, such as a first fluidic channel. In some embodiments, the device comprises a middle chamber. As used herein, the term “middle chamber” is used interchangeably with the term “the chamber”. In some embodiments, the device comprises a first pore connecting the upper chamber and middle chamber. In some embodiments, the device comprises a second pore connecting the middle chamber and a lower chamber. As used herein, the term “lower chamber” is used interchangeably with the term “fluidic channel” and “geometrically constrained fluidic volume”, such as a second fluidic channel. In some embodiments, the device comprises a lower chamber. In some embodiments, the device comprises a second fluidic channel. In some embodiments, the first fluidic volume, the second fluidic volume, the first fluidic channel, the second fluidic channel, and/or the chamber contain one or more electrodes for connecting to a power supply so that a separate voltage can be established across each of the pores between the chambers. In some embodiments, the device comprises an electrode connected to a power supply configured to provide a first voltage between the first fluidic channel and the chamber of the device, and provides a second voltage between the chamber and a second fluidic channel of the device. In some embodiments, the chamber is positioned above the first and second pores. In some embodiments, the chamber is positioned above the first and second fluidic channels. In some embodiments, the chamber is positioned below the first and second pores. In some embodiments, the chamber is positioned between the first and second pores. In some embodiments, the chamber is positioned between the first and second fluidic channels.

In some cases, the shape of the first and/or second fluidic channels can be circular, square, rectangular, hexagonal, triangular, oval, polygon, V-shape, U-shape, or any other suitable shape. In some cases, the first fluidic channel and the second fluidic channel each have a V-shape and each have openings on either end of the V-shape, the V-shapes of the first and second fluidic channels arranged on the chip opposite one another with points of the V-shapes being adjacent to each other, and wherein the first nanopore is positioned at the point of the V-shape of the first fluidic channel and the second nanopore is positioned at the point of the V-shape of the second fluidic channel. In some embodiments, each of the fluidic channels is a different shape. The fluidic channels are not limited to the shapes and/or sizes as described herein and can be any shape and/or size as required per conditions specified to its intended use.

In some cases, the fluidic channels of the nanopore device comprises one or more openings on a side opposite of the first and/or second pores. In some cases, the fluidic channels of the nanopore device comprises two openings on a side opposite of the first and/or second pores.

In some embodiments, the nanopore device has electrodes positioned in the fluidic channels, geometrically constrained volume, or chambers and coupled to one or more power supplies in order to apply voltages across the nanopore(s). In some aspects, the power supply includes a voltage-clamp or a patch-clamp, which can supply a voltage across each pore and measure the current through each pore independently. In this respect, the power supply and the electrode configuration can set the chamber to a common ground for both power supplies. As such each nanopore can have its own respective applied voltage.

In some aspects, a first voltage V1 and a second voltage V2 of different nanopores of a nanopore device are independently adjustable. In one aspect, where multiple nanopores are connected by a chamber, the chamber can be adjusted to be a ground relative to the two voltages. In one aspect, the chamber comprises a medium for providing conductance between each of the pores and the electrode in the chamber. In one aspect, the chamber includes a medium for providing a resistance between each of the nanopores and the electrode in the chamber. Keeping such a resistance sufficiently small relative to the nanopore resistances is useful for decoupling the two voltages and currents across the pores, which is helpful for the independent adjustment of the voltages.

Adjustment of the voltages can be used to control the movement of charged particles in the chambers. For instance, when both voltages are set in the same polarity, a properly charged particle can be moved from the first fluidic channel to the chamber and to the second fluidic channel, or the other way around, sequentially. In some aspects, when the two voltages are set to opposite polarity, a charged particle can be moved from either the first fluidic channel or the second fluidic channel to the chamber and kept there.

The adjustment of the voltages in the device can be particularly useful for controlling the movement of a large molecule, such as a charged polymer, that is long enough to cross both pores at the same time. In such an aspect, the direction and the speed of the movement of the molecule can be controlled by the relative magnitude and polarity of the voltages as described below.

In some cases, the first initial voltage ranges from 0 mV to 1000 mV. In some cases, the first initial voltage ranges from 100-200 mV, 200-300 mV, 300-400 mV, 400-500 mV, 500-600 mV, 600-700 mV, 700-800 mV, 800-900 mV, 900-1000 mV, or 1000 or more mV. In some cases, the first initial voltage is 100 mV, 200 mV, 300 mV, 400 mV, 500 mV, 600 mV, 700 mV, 800 mV, 900 mV, or 1000 mV. In some cases, the second initial voltage ranges from 0 mV to 1000 mV. In some cases, the second initial voltage ranges from 100-200 mV, 200-300 mV, 300-400 mV, 400-500 mV, 500-600 mV, 600-700 mV, 700-800 mV, 800-900 mV, 900-1000 mV, or 1000 or more mV. In some cases, the second initial voltage is 100 mV, 200 mV, 300 mV, 400 mV, 500 mV, 600 mV, 700 mV, 800 mV, 900 mV, or 1000 mV.

In some cases, the methods of the present disclosure comprise adjusting the first and/or second voltages to control the movement of the target polynucleotide in the first pore, the first fluidic channel, the second pore, the second fluidic channel, and/or the chamber of the device. In some cases, the first voltage is adjusted to 0 mV after the target polynucleotide moves from the chamber, through the first pore, and into the first fluidic channel. In some cases, the first voltage is adjusted to 0 mV before translocation through the first pore, wherein at least a portion of the target polynucleotide is positioned in the chamber and at least a portion of the target polynucleotide is positioned in the first fluidic channel. In some cases, the second voltage at the second pore is adjusted to 500 mV when at least a portion of the target polynucleotide is positioned in the chamber and at least a portion of the target polynucleotide is positioned in the chamber. In some cases, the first voltage is adjusted to 0 mV, 50 mV, 100 mV, 150 mV, 200 mV, 250 mV, 300 mV, 350 mV, 400 mV, 450 mV, 500 mV, 550 mV, or 600 mV in the first direction, the second direction, the third direction, and/or the fourth direction. In some cases, the second voltage is adjusted to 0 mV, 50 mV, 100 mV, 150 mV, 200 mV, 250 mV, 300 mV, 350 mV, 400 mV, 450 mV, 500 mV, 550 mV, or 600 mV in the first direction, the second direction, the third direction, and/or the fourth direction. In some cases, the first voltage is adjusted to an intermediate voltage of 0 mV, and the second voltage is adjusted to 500 mV in in the third direction (e.g. when at least a portion of the target polynucleotide is co-captured in the first pore and the second pore). In some cases, the first voltage is adjusted to 400 mV, and the second voltage is adjusted to 500 mV in the third direction (e.g. when at least a portion of the target polynucleotide is cocaptured in the first pore and the second pore). In some cases, the first voltage is adjusted to a voltage of 200 mV, and the second voltage is adjusted to a voltage of 500 mV in the third direction (e.g. when at least a portion of the target polynucleotide is co-captured in the first pore and the second pore).

In some embodiments, a charged polymer, such as a polynucleotide, has a length that is longer than the combined distance that includes the depth of both pores plus the distance between the two pores. For example, a 1000 bp dsDNA is ˜340 nm in length, and would be substantially longer than the 40 nm spanned by two 10 nm-length pores separated by 20 nm. In a first step, the polynucleotide is loaded into either the first fluidic channel or the second fluidic channel. In a first step, the polynucleotide is loaded into the chamber (e.g. the middle chamber or common chamber) of the device. By virtue of its negative charge under a physiological condition (˜pH 7.4), the polynucleotide can be moved across a pore on which a voltage is applied. Therefore, in a second step, two voltages, in the same direction and at the same or similar magnitudes, are applied to the pores to induce movement of the polynucleotide across both pores sequentially. At about time when the polynucleotide reaches the second pore, one or both of the voltages can be changed. Since the polynucleotide is longer than the distance covering both pores, when the polynucleotide reaches the second pore, it is also in the first pore. A prompt change of direction of the voltage at the first pore, therefore, will generate a force that pulls the polynucleotide away from the second pore.

In some embodiments, the dual-pore device of the present disclosure can be used to carry our analysis of molecules or particles that move or are kept within the device by virtue of the controlled voltages applied over the pores. In one aspect, the analysis is carried out at either or both of the pores. Each voltage-clamp or patch-clamp system measures the ionic current through each pore, and this measured current is used to detect the one or more features of the passing charged particle or molecules, or any features associated with a passing charged particle or molecule.

As provided above, a polynucleotide can be loaded into both pores by two voltages having the same direction. In this example, once the direction of the voltage applied at the first pore is inversed and the new voltage-induced force is slightly less, in magnitude, than the voltage-induced force applied at the second pore, the polynucleotide will continue moving in the same direction, but at a markedly lower speed. In this respect, the amplifier supplying voltage across the second pore also measures current passing through the second pore, and the ionic current then determines the identification of a nucleotide that is passing through the pore, as the passing of each different nucleotide would give rise to a different current signature (e.g., based on shifts in the ionic current amplitude). Identification of each nucleotide in the polynucleotide, accordingly, reveals the sequence of the polynucleotide.

In some embodiments, the adjusted first voltage and second voltage at step are about 10 times to about 10,000 times as high, in magnitude, as the difference between the two voltages. For instance, the two voltages are 90 mV and 100 mV, respectively. In some embodiments, the magnitude of the voltages (^{˜}100 mV) is about 10 times of the difference between them, 10 mV. In some embodiments, the magnitude of the voltages is at least about 15 times, 20 times, 25 times, 30 times, 35 times, 40 times, 50 times, 100 times, 150 times, 200 times, 250 times, 300 times, 400 times, 500 times, 1000 times, 2000 times, 3000 times, 4000 times, 5000 times, 6000 times, 7000 times, 8000 times or 9000 times as high as the difference between them. In some aspects, the magnitude of the voltages is no more than about 10000 times, 9000 times, 8000 times, 7000 times, 6000 times, 5000 times, 4000 times, 3000 times, 2000 times, 1000 times, 500 times, 400 times, 300 times, 200 times, or 100 times as high as the difference between them.

In some aspects, repeated controlled delivery for re-sequencing a polynucleotide, for instance, with respect to enrichment of target material from a sample, further improves the quality of sequencing. Each voltage is alternated as being larger, for controlled delivery in each direction.

The device can contain materials suitable for holding liquid samples, in particular, biological samples, and/or materials suitable for nanofabrication. In one aspect, such materials include dielectric materials such as, but not limited to, silicon, silicon nitride, silicon dioxide, graphene, carbon nanotubes, TiO2, HfO2, Al2O3, or other metallic layers, or any combination of these materials. In some aspects, for example, a single sheet of graphene membrane of about 0.3 nm thick can be used as the pore-bearing membrane.

Nanopore devices that are microfluidic can be made by a variety of means and methods. A focused electron or ion beam can be used to drill pores through the membranes, naturally aligning them. The pores can also be sculpted (shrunk) to smaller sizes by applying a correct beam focusing to each layer. Any single nanopore drilling method can also be used to drill the pair of pores in the two membranes, with consideration to the drill depth possible for a given method and the thickness of the membranes. Predrilling a micro-pore to a prescribed depth and then a nanopore through the remainder of the membranes is also possible to further refine the membrane thickness. In one example, a single beam can be used to form one or more nanopores (e.g., concentric nanopores) in a membrane of the nanopore device. Alternatively, in another example, different beams can be applied to each side of a on each side of the membranes, in order to generate aligned or non-aligned nanopores.

More specifically, the nanopore-bearing membranes can be made with transmission electron microscopy (TEM) grids with a 5-100 nm thick silicon, silicon nitride, or silicon dioxide windows. Spacers can be used to separate the membranes, using an insulator, such as SU-8, photoresist, PECVD oxide, ALD oxide, ALD alumina, or an evaporated metal material, such as Ag, Au, or Pt, and occupying a small volume within the otherwise aqueous portion of a middle chamber (e.g. chamber).

By virtue of the voltages present at the pores of the device, charged molecules can be moved through the pores between chambers. Speed and direction of the movement can be controlled by the magnitude and polarity of the voltages. Further, because each of the two voltages can be independently adjusted, the direction and speed of the movement of a charged molecule can be finely controlled in each chamber. For example, when a first set of features are detected in a first cycle in a first direction, the first voltage, the second voltage, or both, can be adjusted to a first and second pore to change the direction of the target molecule moves from the second pore to the first pore in a second direction.

In some aspects, a nanopore device further includes means to move a polymer across the pore and/or means to identify objects that pass through the pore. In some embodiments, the polymer is a polynucleotide or a polypeptide. In some aspects, the polymer is a polynucleotide. Non-limiting examples of polynucleotides include double-stranded DNA, single-stranded DNA, double-stranded RNA, single-stranded RNA, and DNA-RNA hybrids.

In some aspects, the dual-pore device can be used to identify one or more features of a polymer. In some embodiments, the one or more features is one feature, two features, three features, four features, or five features. In some embodiments, the one or more features is two or more features, three or more features, four or more features, five or more features, six or more features, seven or more features, eight or more features, nine or more features, or ten or more features. In some embodiments, the one or more features ranges from 1-5 features, 5-10 features, 10-15 features, 15-20 features, 20-25 features, 25-30 features, 30-35 features, 35-40 features, 40-45 features, or 45-50 features. In some embodiments, the one or more features ranges from 50 features to 100 features, 100 features to 1,000 features, 1,000 features to 10,000 features, 10,000 features to 100,000, 100,000 features to 200,000 features. In some embodiments, the one or more features is 50 features or more, 100 features or more, 1,000 features or more, 10,000 features or more, 100,000 features or more, or 200,000 features or more.

Aspects of the present disclosure include one or more features, wherein each feature is about from one another by about 100 base pairs, 300 base pairs, 500 base pairs, 1 kilo-base pair, 5 kilo base-pair, 10 kilo base pair, 20 kilo-base pair, or a combination thereof. In some embodiments, each features is spaced about from one another by about 25 base pairs or more, about 50 base pairs or more, about 100 base pairs or more, about 300 base pairs or more, about 500 base pairs or more, about 1 kilo-base pair or more, about 5 kilo base-pairs or more, about 10 kilo base pairs or more, about 20 kilo-base pairs or more, or a combination thereof. In some embodiments, each features is spaced about from one another by about 25 base pairs or less, about 50 base pairs or less, about 100 base pairs or less, about 300 base pairs or less, about 500 base pairs or less, about 1 kilo-base pair or less, about 5 kilo base-pairs or less, about 10 kilo base pairs or less, about 20 kilo-base pairs or less, or a combination thereof.

In some aspects, the dual-pore device can be used to identify a first set of features, a second set of features, a third set of features, a fourth set of features, a fifth set of features, a sixth set of features, a seventh set of features, an eighth set of features, a ninth set of features, and/or a tenth set of features. In some cases, each set of features comprises one or more features ranges from 1-5 features, 5-10 features, 10-15 features, 15-20 features, 20-25 features, 25-30 features, 30-35 features, 35-40 features, 40-45 features, or 45-50 features. In some embodiments, the first set of features overlaps with the second set of features. In some embodiments, the third set of features overlaps with the fourth set of features. In some embodiments, the first set of features partially overlaps with the second set of features. In some embodiments, the third set of features partially overlaps with the fourth set of features. In some embodiments, the first set of features are the same as the second set of features. In some embodiments, the third set of features are the same as the fourth set of features. In some embodiments, the first set of features are different from the second set of features. In some embodiments, the third set of features are different from the fourth set of features.

In some embodiments, the sets of features (e.g. first set, second set, third set, fourth set, fifth set, sixth set, seventh set, eighth set, ninth set, and/or tenth set) are associated with a first cycle, a second cycle, a third cycle, a fourth cycle, a fifth cycle, a sixth cycle, a seventh cycle, an eighth cycle, a ninth cycle, and/or a tenth cycle, respectively. In some cases, a first cycle comprises one or more scans performed by a processor to detect the first set of features. In some cases, the first cycle comprises two or more scans, three or more scans, four or more scans, five or more scans, six or more scans, seven or more scans, eight or more scans, nine or more scans, or ten or more scans. In some cases, the first cycle comprises two or more scans, four or more scans, six or more scans, eight or more scans, ten or more scans, twelve or more scans, fourteen or more scans, sixteen or more scans, eighteen or more scans, or twenty or more scans. In some cases, the first cycle comprises five or more scans, ten or more scans, fifteen or more scans, twenty or more scans, twenty-five or more scans, thirty or more scans, thirty-five or more scans, forty or more scans, forty-five or more scans, or fifty or more scans.

In some cases, the second cycle comprises one or more scans performed by a processor to detect the third set of features. In some cases, the second cycle comprises two or more scans, three or more scans, four or more scans, five or more scans, six or more scans, seven or more scans, eight or more scans, nine or more scans, or ten or more scans. In some cases, the second cycle comprises two or more scans, four or more scans, six or more scans, eight or more scans, ten or more scans, twelve or more scans, fourteen or more scans, sixteen or more scans, eighteen or more scans, or twenty or more scans. In some cases, the second cycle comprises five or more scans, ten or more scans, fifteen or more scans, twenty or more scans, twenty-five or more scans, thirty or more scans, thirty-five or more scans, forty or more scans, forty-five or more scans, or fifty or more scans. In some cases, the first cycle and the second cycle, together, comprise 50 or more scans, 100 or more scans, 150 or more scans, 200 or more scans, 250 or more scans, 300 or more scans, 350 or more scans, 400 or more scans, or 500 or more scans. In some embodiments, the first cycle, second cycle, third cycle, fourth cycle, and fifth cycle, together, comprise 50 or more scans, 100 or more scans, 150 or more scans, 200 or more scans, 250 or more scans, 300 or more scans, 350 or more scans, 400 or more scans, or 500 or more scans.

Aspects of the present disclosure include a processor and a computer-readable medium, comprising instructions that cause the processor to repeat the determining the presence of the target polynucleotide in both pores, scanning for one or more features, and changing the voltage to control movement of the polynucleotide (e.g. in either direction) for a third cycle, a fourth cycle, and a fifth cycle; or when the polynucleotide exits the device, or otherwise enters a chamber of the device for retrieval and/or subsequent downstream processing.

In some aspects, the dual-pore device can be used to identify one or more features of a polymer. In some embodiments, the polymer is a polynucleotide. In some embodiments, the one or more features of the polynucleotide comprises one or more features associated with the polynucleotide. Non-limiting examples of one or more features associated with the polynucleotide, include, but are not limited to, transcription factors, nucleosomes, or modifications to the features, including modification to histone tails. In some embodiments, one or more features in the polynucleotide comprises one or more sequence or structural variations.

In some embodiments, the one or more features of the polynucleotide comprises one or more payload molecules bound to the polynucleotide. In some embodiments, the one or more features of the polynucleotide comprises one or more payload molecules hybridized to the polynucleotide. In some embodiments, the one or more features of the polynucleotide comprises one of more payload molecules incorporated into the genome of the polynucleotide. In some embodiments, the one or more features of the polynucleotide comprises a molecular motif on a polynucleotide sequence of the target polynucleotide. In some embodiments, the one or more features comprises the position of: one or more CpG's; or one or more methylation cites and CpG's, on the polynucleotide sequence of the target polynucleotide. In some embodiments, the one or more features comprises the position of one or more histones on the target polynucleotide. In some embodiments, the one or more features comprises a molecule selected from the group consisting of: a nucleic acid, a TALEN, a CRISPR, a peptide nucleic acid, and a chemical compound. In some embodiments, the one or more features comprises a DNA-binding protein, a polypeptide, an anti-DNA antibody, a streptavidin, a transcription factor, a histone, a peptide nucleic acid (PNA), a DNA-hairpin, a DNA molecule, an aptamer, or a combination thereof.

Non-limiting examples of payload molecules bound to the polynucleotide can be found in can be found in U.S. Patent Publication No. 2018/0023115, which is hereby incorporated by reference in its entirety. For example, a payload molecule can include a dendrimer, double stranded DNA, single stranded DNA, a DNA aptamer, a fluorophore, a protein, a polypeptide, a nanorod, a nanotube, fullerene, a PEG molecule, a liposome, or a cholesterol-DNA hybrid. In some embodiments, the polynucleotide and the payload are connected directly or indirectly via a covalent bond, a hydrogen bond, an ionic bond, a van der Waals force, a hydrophobic interaction, a cation-pi interaction, a planar stacking interaction, or a metallic bond. The payload adds size to the target polynucleotide or amplicon, and facilitates detection, with the amplicon bound to the payload having a markedly different current signature when passing through the nanopore than background molecules. In some embodiments, the payload molecule comprises an azide chemical handle for attachment to a primer. In some embodiments, the primer is bound to a biotin molecule. In some embodiments, the payload molecule can bind to another molecule to affect the bulkiness of the molecule, thereby enhancing the sensitivity of detection of the amplicon in a nanopore. In some embodiments, the primer is bound to or comprises a binding site for binding to a biotin molecule. In some embodiments, the biotin is further bound by streptavidin to increase the size of the payload molecule for enhanced detection in a nanopore over background molecules. The added bulk can produce a more distinct signature difference between amplicon comprising a target sequence and background molecules.

In this embodiment, attachment of a payload to a primer or amplicon can be achieved in a variety of ways. For example, the primer may be a dibenzocyclooctyne (DBCO) modified primer, effectively labeling all amplicons with a DBCO chemical group to be used for conjugation purposes via copper-free “click” chemistry to an azide-tagged amplicon or primer.

In some aspects, the primer comprises a chemical modification that causes or facilitates recognition and binding of a payload molecule. For example, methylated DNA sequences can be recognized by transcription factors, DNA methyltransferases or methylation repair enzymes. In other embodiments, biotin may be incorporated into, and recognized by, avidin family members. In such embodiments, biotin forms the fusion binding domain and avidin or an avidin family member is the polymer scaffold-binding domain on the fusion. Due to their binding complementarity, payload molecule binding domains on a primer/amplicon and primer binding domains on a payload molecule may be reversed so that the payload binding domain becomes the primer binding domain, and vice versa.

Molecules, in particular, proteins, that are capable of specifically recognizing nucleotide binding motifs are known in the art. For instance, protein domains such as helix-turn-helix, a zinc finger, a leucine zipper, a winged helix, a winged helix turn helix, a helix-loop-helix and an HMG-box, are known to be able to bind to nucleotide sequences. Any of these molecules may act as a payload molecule binding to the amplicon or primer. In some aspects, the payload binding domains can be locked nucleic acids (LNAs), bridged nucleic acids (BNA), Protein Nucleic Acids of all types (e.g. bisPNAs, gamma-PNAs), transcription activator-like effector nucleases (TALENs), clustered regularly interspaced short palindromic repeats (CRISPRs), or aptamers (e.g., DNA, RNA, protein, or combinations thereof).

In some aspects, the payload binding domains are one or more of DNA binding proteins (e.g., zinc finger proteins), antibody fragments (Fab), chemically synthesized binders (e.g., PNA, LNA, TALENS, or CRISPR), or a chemical modification (i.e., reactive moieties) in the synthetic polymer scaffold (e.g., thiolate, biotin, amines, carboxylates).

In some embodiments, the one or more features comprises one or more features in the polynucleotide. In some embodiments, the one or more features in the polynucleotide comprises one or more modifications to the polynucleotide. In some embodiments, the one or more modifications comprises DNA methylation (e.g. 5mC, 5hmC, e.g., at CpG dinucleotides, 5 mA, and the like). In some embodiments, the one or more features in the polynucleotide comprise sequence variations, mutations, or larger structural variations. In some embodiments, the one or more features in the polynucleotide comprises rearrangements, deletions, insertions, and/or translocations to the polynucleotide sequence.

In some embodiments, the one or more features comprises one or more features on the polynucleotide. In some embodiments, the one or more features on the polynucleotide comprises a modification to the polynucleotide. In some embodiments, the modification comprises a molecule bound to a monomer. In some embodiments, the one or more features on the polynucleotide comprises one or more molecules bound to the polynucleotide. In some embodiments, the modification comprises the binding of a molecule to the polynucleotide. For instance, for a DNA molecule, the bound molecule can be a DNA-binding protein, such as RecA, NF-κB and p53. In some embodiments, the modification is a particle that binds to a particular monomer or fragment. For instance, quantum dots or fluorescent labels bound to a particular DNA site for the purpose of genotyping or DNA mapping can be detected by the device.

In some embodiments, the polynucleotide sequence comprises one or more nick sites. As a non-limiting example, a nicking restriction endonuclease introduces a nick at the recognition sequence for bar coding. This sequence appears many times in a genome. A single azide azide N3 labeled nucleotide is introduced at the nick site. The reaction is filtered to remove unincorporated nucleotide. A DNA molecule labeled with a DCBO either 5′, 3′, or body labeled is added to the reaction. The DNA molecule is covalently linked at the nick site via copperless click chemistry. 1000-10000 fold excess DNA molecule can be used. In another non-limiting example, a Cas9 D10A nickase can be used for site-specific labeling. Cas9-D10A is target to a specific site and a single strand nick is introduced. Cas9 D10A is removed. A single azide N3 nucleotide is introduced at the nick site by nick translation. The reaction is filtered to remove unincorporated nucleotide. A DNA molecule labeled with a DCBO either 5′, 3′, or body labeled is added to the reaction. The DNA molecule is covalently linked at the nick site via copperless click chemistry. 1000-10000 fold excess DNA molecule can be used.

In one embodiment, a nanopore device includes a plurality of chambers, each chamber in communication with an adjacent chamber through at least one pore.

In some embodiments, a nanopore device can be a multi-pore device having more than one pore. In some embodiments, a nanopore device can include two nanopores, where a first nanopore is positioned relative to a second nanopore in a manner in order to allow at least a portion of a target polynucleotide to move out of the first nanopore and into the second nanopore. In some embodiments, the nanopore device includes one or more sensors at each nanopore, where a respective sensor is capable of identifying a target polynucleotide during the movement across at least one of the nanopores. In some embodiments, the identification entails identifying individual components of the target polynucleotide. In some embodiments, the identification entails identifying payload molecules bound to the target polynucleotide. When a single sensor is employed, the single sensor may include two electrodes placed at both ends of a pore to measure an ionic current across the pore. In another embodiment, the single sensor comprises a component other than electrodes.

In some embodiments, a nanopore device includes three chambers connected through two pores. Devices with more than three chambers can be readily designed to include one or more additional chambers on either side of a three-chamber device, or between any two of the three chambers. Likewise, more than two nanopores can be included in the device to connect the chambers. In some embodiments, the chamber is connected to a common ground relative to the two voltages.

In one aspect, there can be two or more pores between two adjacent chambers, to allow multiple polymer scaffolds to move from one chamber to the next simultaneously. Such a multi-pore design can enhance throughput of target polynucleotide analysis in the device. For multiplexing, one chamber could have a one type of target polynucleotide, and another chamber could have another target polynucleotide type.

In some aspects, the device further includes means to move a target polynucleotide from one chamber to another. In one aspect, the movement results in loading the target polynucleotide (e.g., the amplification product or amplicon comprising the target sequence) across both the first pore and the second pore at the same time. In another aspect, the means further enables the movement of the target polynucleotide, through both pores, in the same direction.

While some variations of nanopore devices are described above, the nanopore device(s) can be configured as described in U.S. Application Publication. No. 2013-0233709, U.S. Pat. No. 9,863,912, and PCT Application Publication No. WO2018/236673, which are hereby incorporated by reference in their entirety.

### Systems and Devices—Sensors

As discussed above, in various aspects, the nanopore device further includes one or more sensors that generate electrical signals corresponding to materials passing through a nanopore.

The sensors used in a nanopore device can include any sensor suitable for identifying a target polynucleotide amplicon bound or unbound to a payload molecule. For instance, a sensor can be configured to identify the target polynucleotide by measuring a current, a voltage, a pH value, an optical feature, or residence time associated with the polymer. In other aspects, the sensor may be configured to identify one or more individual components of the target polynucleotide or one or more components bound or attached to the target polynucleotide. The sensor may be formed of any component configured to detect a change in a measurable parameter where the change is indicative of the target polynucleotide, a component of the target polynucleotide, or in some cases, a component bound or attached to the target polynucleotide. In one aspect, the sensor includes a pair of electrodes placed at two sides of a pore to measure an ionic current across the pore when a molecule or other entity, in particular a target polynucleotide, moves through the pore. In certain aspects, the ionic current across the pore changes measurably when a target polynucleotide segment passing through the pore is bound to a payload molecule. Such changes in current may vary in predictable, measurable ways corresponding with, for example, the presence, absence, and/or size of the target polynucleotide molecule present.

In one embodiment, the sensor comprises electrodes that apply voltage and are used to measure current across the nanopore. Translocations of molecules through the nanopore provides electrical impedance (Z) which affects current through the nanopore according to Ohm's Law, V=IZ, where V is voltage applied, I is current through the nanopore, and Z is impedance. Inversely, the conductance G=1/Z are monitored to signal and quantitate nanopore events. The result when a molecule translocates through a nanopore in an electrical field (e.g., under an applied voltage) is a current signature that may be correlated to the molecule passing through the nanopore upon further analysis of the current signal.

When residence time measurements from the current signature are used, the size of the component can be correlated to the specific component based on the length of time it takes to pass through the sensing device.

In one embodiment, a sensor is provided in the nanopore device that measures an optical feature of the polymer, a component (or unit) of the polymer, or a component bound or attached to the polymer. One example of such measurement includes the identification of an absorption band unique to a particular unit by infrared (or ultraviolet) spectroscopy.

In some embodiments, the sensor is an electric sensor. In some embodiments, the sensor detects a fluorescent signature. A radiation source at the outlet of the pore can be used to detect that signature. Non-limiting examples of sensor circuitry in the nanopore device can be found in PCT Application Publication No. WO/2018/236673, which is hereby incorporated by reference in its entirety.

### Systems and Devices—Processor, Controller, and Other Elements

As described above, embodiments system of the present disclosure are configured to interface with the set of one or more nanopore devices and include an electronics subsystem for receiving electrical signals from the sensors of the set of nanopore devices and for sorting material (e.g., target material, non-target material) of a sample based upon the received electrical signals. The electrical subsystem can include signal processing elements (e.g., amplifiers, filters, signal pre-conditioning elements, etc.) and/or elements for controlling voltage applied across different nanopores, in order to enable automated detection and sorting of sample material using the nanopore device.

Aspects of the present disclosure includes a device comprising a processor. In some embodiments, the device comprises a non-transitory computer-readable medium comprising instructions that cause the processor to determine, from the one or more sensors, the simultaneous presence of the target polynucleotide in one or more of the multiple pores of the nanopore device. In some embodiments, the instructions cause the processor to scan for one or more features of the target polynucleotide. In some embodiments, the instructions cause the processor to measure or detect the first set of features in the first cycle in the first direction, and, responsive to that count, adjust one or both of the first and second voltages, to produce a first force and an opposing second force acting on said target polynucleotide. In some embodiments, the first and second forces change the direction and the speed of the movement of the target polynucleotide so that at least a portion of the target polynucleotide moves from the second pore to the first pore in the second direction. In some embodiments, the process is repeated to detect a second set of features, in a second cycle. In some embodiments, the process to detect third and fourth sets of features, in a second cycle. In some embodiments, the steps are repeated until the polynucleotide exits the dual-pore device.

In some embodiments, the computer-readable medium further comprises instructions that cause the processor to detect signatures associated with target material and non-target material of a sample, and to generate control instructions for directing target material and/or non-target material to portions (e.g., a second nanopore, a chamber that can be flushed, etc.) of the nanopore device for downstream processing. In variations, the processor can further generate control instructions for one or more of: enabling removal of non-target material from the device (e.g., with flushing of a chamber of the device into which non-target material has been directed); re-processing non-removed material from the device, thereby sorting target material from non-target material in a second run; delivering an enriched volume of target material from the device for downstream processing; amplifying target material (e.g., within the device, outside of the device); generating analyses characterizing aspects of the sample with respect to target material and non-target material composition; and performing other suitable functions.

In some embodiments, the processor can further comprise architecture for implementing machine learning algorithms that are trained to detect one or more features of target material and/or non-target material of a sample based on training data and probabilistic models, that will be described in further detail below.

Aspects of the present disclosure include a device that comprises a controller. In some embodiments, the controller is a field programmable gate array (FPGA). In some embodiments, the controller is configured to control the number of features to scan for. In some embodiments, the controller is configured to control the number of features to re-scan. In some embodiments, the controller is configured to control the movement of the target polynucleotide. In some embodiments, the controller is configured to control the direction of the target polynucleotide. In some embodiments, the controller determines which of the one or more features to perform additional scans on. In some embodiments, the controller determines when to move away from one or more features already detected. In some embodiments, the controller determines when to scan for regions on the polynucleotide that have not yet been scanned. In some embodiments, the FPGA executes control logic to change the: a) number of features to scan for; b) number of features to re-scan; c) movement or direction of the target polynucleotide; d) direction of the target polynucleotide; or e) a combination thereof.

In some embodiments, the processor and computer-readable medium comprising instructions cause the processor to carry out the functions instructed by the controller (e.g. number of features to scan for; number of features to re-scan; movement of a target polynucleotide for sorting; movement of a non-target polynucleotide for sorting; direction of the target polynucleotide; and/or a combination thereof). In some embodiments, the processor is a field programmable gate array (FPGA) or an application-specific integrated circuit (ASIC).

In some embodiments, the controller, a processor, and a non-transitory computer-readable medium comprising instructions that cause the processor to: change the direction of the target polynucleotide when a target (e.g., barcode sequence, other target) is detected. In some embodiments, the first voltage and the second voltage is adjusted in real-time, wherein said adjusting is performed by an active feedback controller using hardware and software. In some embodiments, the controller is configured to control the first or second voltage based on feedback of the first or second or both ionic current measurements.

Embodiments of the device and system can also include a processor including architecture with logic for implementing a set of operation modes including a first operation mode for measuring and evaluating a set of metrics derived from received electrical signals associated with one or more features of the molecule, a second operation mode for generating an assessment of the one or more features upon processing values of the set of metrics, and a third operation mode for executing one or more actions to continue scanning the same region of the molecule to search for additional features, continue scanning the same region of the molecule for re-scanning of the same probes already detected, vary the number of probes to scan in the same region, or move to a different region of the molecule for scanning, based upon the assessment. As such, the system can include structures for implementing embodiments of the method(s) described in more detail below.

The device and system can also generate notifications for provision to an operator of the system. The notifications can include content describing one or more of: a status of the system a status of one or more nanopore devices interfacing with control elements of the system, a status of one or more nanopores, instructions for adjusting operation of the system, instructions for proceeding with an experimental protocol in relation to nanopore/nanopore device status, and other content. The notifications can be rendered by the system in a visual format (e.g., using a display), an audible format (e.g., using a speaker), haptically (e.g., using a haptic device), and/or in another other suitable format.

The device and system can also generate computer-readable instructions for transitioning between different system operation modes (e.g., transitioning to an idle mode, transitioning to a “stop experiment” mode, transitioning to a “resume experiment” mode, transitioning to a calibration mode, transitioning to a mode involving use of a subset of nanopores still having suitable quality, etc.) in relation to nanopore/nanopore device status. The computer-readable instructions can be transmitted to a controller of the system, in order to transition the system between operation modes.

An embodiment of a machine learning architecture associated with embodiments of the systems and methods described “learns” when to move from one location to another on a target polynucleotide, when to continuously scan one or more features, when to vary the number of features to scan, and when to switch from continuously scanning one or more features to moving further away from the one or more features already scanned to a location that has not yet been surveyed/scanned, in a polynucleotide. The automation goal is to generate a sufficiently informative data set in order to build a consensus map for each molecule (i.e. polynucleotide). For example, a machine learning architecture with control logic can provide for scanning a region of a molecule for a period of time, build a local map of that region in real-time, and then move to a different location that has not yet been scanned to build a consensus map for the molecule. In an example, Bayesian Optimization, which is operable on hardware with limited processing power that needs to react at/near real time can be used. While Bayesian optimization is described, other statistical and/or machine learning approaches can be used to for automated detection of features associated with target material of a sample. In variations, such models can implement a learning style including unsupervised learning (e.g., using K-means clustering), supervised learning (e.g., using regression, using back propagation networks), semi-supervised learning, reinforcement learning, or any other suitable learning style.

The device and system can additionally or alternatively implement any one or more of: a regression algorithm (e.g., least squares, logistic, stepwise, multivariate adaptive, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method, a kernel method (e.g., a support vector machine, a radial basis function, a linear discriminate analysis, etc.), a clustering method (e.g., k-means clustering, expectation maximization, etc.), an associated rule learning algorithm (e.g., an Eclat algorithm, etc.), a neural network, a deep learning algorithm, a dimensionality reduction method (e.g., principal component analysis, partial lest squares regression, etc.), an ensemble method (e.g., boosting, bootstrapped aggregation, AdaBoost, stacked generalization, gradient boosting machine method, random forest method, etc.), and any suitable form of algorithm.

Applications of such algorithms for automated searching and surveying for map generation of a molecule, are described in more detail below.

In some aspects, the device and systems of the present disclosure include a non-transitory computer-readable medium, comprising instructions that cause a processor to: i) determine, from the sensor, the simultaneous presence of the target polynucleotide in both pores; ii) scan for one or more features of the target polynucleotide; iii) count the first set of features in the first cycle in the first direction, and, responsive to that count, adjust one or both of the first and second voltages, to produce a first force and an opposing second force acting on said target polynucleotide, wherein said first and second forces change the direction and the speed of the movement of the target polynucleotide so that at least a portion of the target polynucleotide moves from the second pore to the first pore in the second direction; and optionally iv) repeat steps i) through iii).

Aspects of the present disclosure include a device for carrying out the functions of the methods described herein. The present disclosure includes a device for mapping one or more features of a polynucleotide sequence of a target polynucleotide through a first and a second pore, the device comprising: (i) an electrode connected configured to provide a first voltage at the first pore of the device, and provide a second voltage at the second pore of the device; (ii) a first pore; (iii) a second pore; wherein the first pore and the second pore are configured such that the target polynucleotide is capable of simultaneously moving across both pores in a first direction or a second direction, and in a controlled manner; (iv) one or more sensors capable of identifying: a first set of features, in a first cycle, from the target polynucleotide, during movement of the target polynucleotide through the first pore and the second pore in the first direction and, a second set of features, in the first cycle, from the target polynucleotide, during movement of the target polynucleotide through the second pore and the first pore in the second direction; (v) a processor; and (vi) a non-transitory computer-readable medium comprising instructions that cause the processor to: a) determine, from the one or more sensors, the simultaneous presence of the target polynucleotide in both pores; b) scan for one or more features of the target polynucleotide; c) count the first set of features in the first cycle in the first direction, and, responsive to that count, adjust one or both of the first and second voltages, to produce a first force and an opposing second force acting on said target polynucleotide, wherein said first and second forces change the direction and the speed of the movement of the target polynucleotide so that at least a portion of the target polynucleotide moves from the second pore to the first pore in the second direction; and d) optionally repeat steps a) through c). In some cases, the instructions further cause the processor to repeat c) until the target polynucleotide enters a chamber for retrieval or otherwise exits the device. In some cases, the first pore and the second pore are about 10 nm to about 2 μm apart from each other.

In some cases, the diameter of the pores ranges from about 2 nm to about 50 nm. In some cases, the diameter of the pore is about 20 nm. In some cases, the diameter of the first and/or second pore ranges from about 2 nm to about 50 nm. In some cases, the diameter of the first and/or second pore ranges from about 2 nm to about 8 nm. In some cases, the diameter of the first and/or second pore ranges from about 10 nm to about 20 nm. In some cases, the diameter of the pore ranges from about 20 nm to about 30 nm. In some cases, the diameter of the first and/or second pore ranges from about 30 nm to about 40 nm. In some cases, the diameter of the first and/or second pore ranges from about 40 nm to about 50 nm. In some cases, the diameter of the first and/or second pore is about 2 nm, about 4 nm, about 6 nm, about 8 nm, about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm. In some cases, the diameter of the first and/or second pore is about 19 nm. In some cases, the first pore and the second pore have the same diameters. In some cases, the diameter of the first and/or second pore is about 21 nm. In some cases, the diameter of the first and/or second pore is about 22 nm. In some cases, the diameter of the first and/or second pore is about 23 nm. In some cases, the diameter of the first and/or second pore is about 24 nm. In some cases, the diameter of the first and/or second pore is about 25 nm. In some cases, the diameter of the first and/or second pore is about 27 nm. In some cases, the diameter of the first and/or second pore is about 29 nm. In some cases, the first pore and the second pore have different diameters. In some cases, the diameter of the pore is about 20 nm.

In some cases, the first pore and the second pore are about 500 nm apart from each other. In some cases, the first pore has a depth of at least about 0.3 nm separating the first channel and the chamber and the second pore has a depth of at least about 0.3 nm separating the chamber and the second channel. In some cases, the chamber is connected to a common ground relative to the two voltages.

In some cases, the device further comprises a controller. In some cases, the controller is configured to vary the number of features of the polynucleotide to scan. In some cases, the controller is configured to vary the number of scans. In some cases, the controller is configured to control the location of the polynucleotide that is scanned. In some cases, the controller is configured to change the region of the polynucleotide that is scanned. In some cases, the controller is configured to control the: a) number of features to scan for; b) number of features to re-scan; c) type of features to scan or re-scan for; d) number of cycles to scan or re-scan for; e) movement of the target polynucleotide; f) direction of the target polynucleotide; g) speed of the target polynucleotide; h) voltage of the first and second pore; or i) a combination thereof.

In some cases, the processor comprises a field programmable gate array (FPGA) or an application-specific integrated circuit (ASIC). In some cases, the controller comprises a field programmable gate array (FPGA) or an application-specific integrated circuit (ASIC). In some cases, the controller is a microcontroller.

In some cases, the device further comprises instructions that cause the processor to compute the distances between features from the speed of a feature of the target polynucleotide, from the time between features detected in the current signal from the first pore, the second pore, or both. In some cases, the device further comprises instructions that cause the processor to compute the speed of a feature of the target polynucleotide for every scan, and to compute statistics on the speed of the feature by using the distribution of speeds. In some cases, the device further comprises instructions that cause the processor to combine the speed of all the features and compute the time history of the speed of the polynucleotide in a given scan and given direction of scanning.

In some cases, the device further comprises instructions that cause the processor to perform a frequency sweep of the polynucleotide in the first direction, second direction, or both. In some cases, the device further comprises instructions that cause the processor to perform an amplitude sweep of the polynucleotide in the first direction, second direction, or both. In some cases, the device further comprises instructions that cause the processor to adjust the speed of the polynucleotide. In some cases, wherein the speed ranges from 1 base pair per millisecond to 10 base pairs per millisecond.

In some cases, the device further comprises instructions that cause the processor to adjust the first and second voltages in order to perform a plurality of scans of the polynucleotide at a plurality of speeds. In some cases, said performing the plurality of scans of the polynucleotide at the plurality of speeds improves the accuracy of the detection of one or more features. In some cases, the device further comprises instructions that cause the processor perform a plurality of scans of the polynucleotide at a plurality of speeds. In some cases, the device further comprises instructions that cause the processor to control the speed range of the polynucleotide in the first direction, second direction, or both. In some cases, the device further comprises instructions that cause the processor to control the voltage range of the first and second pores when the polynucleotide moves through the first and second pore in the first direction, second direction, or both. In some cases, the device further comprises instructions that cause the processor to determine an optimal speed range of the polynucleotide in the first direction, second direction, or both, wherein the optimal speed range of the polynucleotide reduces the effect of Brownian motion on the polynucleotide.

In some cases, controlling the speed range of the polynucleotide comprises determining the optimal speed range of the polynucleotide for sequencing.

In some cases, the target polynucleotide is substantially linearized. In some cases, the target polynucleotide is substantially linearized by the action of the adjustments to the first voltage, or the second voltage, or both.

Aspects of the present disclosure include systems for carrying out the methods disclosed herein. The system comprises a) a dual-pore, dual-amplifier device for mapping one or more features of a polynucleotide sequence of a target polynucleotide through a first and a second pore, the device comprising: (i) an electrode connected to a power supply configured to provide a first voltage at the first pore of the device, and provide a second voltage at the second pore of the device; (ii) a first pore; (iii) a second pore; wherein the first pore and the second pore are configured such that the target polynucleotide is capable of simultaneously moving across both pores in a first direction or a second direction, and in a controlled manner; (iv) one or more sensors capable of identifying: a first set of features, in a first cycle, from the target polynucleotide, during movement of the target polynucleotide through the first pore and the second pore in the first direction and, a second set of features, in the first cycle, from the target polynucleotide, during movement of the target polynucleotide through the second pore and the first pore in the second direction; c) a processor; and d) a non-transitory computer-readable medium, comprising instructions that cause the processor to: i) determine, from the sensor, the simultaneous presence of the target polynucleotide in both pores; ii) scan for one or more features of the target polynucleotide; iii) measure the first set of features in the first cycle in the first direction, and, responsive to that measurement, adjust one or both of the first and second voltages, to produce a first force and an opposing second force acting on said target polynucleotide, wherein said first and second forces change the direction and the speed of the movement of the target polynucleotide so that at least a portion of the target polynucleotide moves from the second pore to the first pore in the second direction; and iv) optionally repeat steps i) through iii) to detect a additional features.

In some cases, the device further comprises a controller. In some cases, the controller is configured to vary the number of features of the polynucleotide to scan. In some cases, the controller is configured to vary the number of scans. In some cases, the controller is configured to control the location of the molecule that is scanned. In some cases, the controller is configured to control the: a) number of features to scan for; b) number of features to re-scan; c) type of features to scan or re-scan for; d) number of cycles to scan or re-scan for; e) movement of the target polynucleotide; f) direction of the target polynucleotide; g) speed of the target polynucleotide; h) voltage of the first and second pore; or i) a combination thereof.

In some cases, the system further comprises instructions that cause the processor to compute the speed of a feature of the target polynucleotide from the time difference between detection of the feature in the first pore and the second pore, and the known distance between pore one and pore two. In some cases, the system further comprises instructions that cause the processor to compute the distances between features from the speed of a feature of the target polynucleotide, from the time between features detected in the current signal from the first pore, the second pore, or both. In some cases, the system further comprises instructions that cause the processor to compute speed of a feature of the target polynucleotide for every scan, and to compute statistics on the speed of the feature by using the distribution of speeds. In some cases, the system further comprises instructions that cause the processor to perform a frequency sweep of the polynucleotide in the first direction, second direction, or both. In some cases, the system further comprises instructions that cause the processor to perform an amplitude sweep of the polynucleotide in the first direction, second direction, or both. In some cases, the system further comprises instructions that cause the processor to adjust the speed of the polynucleotide.

In some cases, the speed ranges from 1 base pair per millisecond to 10 base pairs per millisecond.

In some cases, the system further comprises instructions that cause the processor to adjust the first and second voltages in order to perform a plurality of scans of the polynucleotide at a plurality of speeds. In some cases, performing the plurality of scans of the polynucleotide at the plurality of speeds improves the accuracy of the detection of one or more features.

In some cases, the system further comprises instructions that cause the processor perform a plurality of scans of the polynucleotide at a plurality of speeds. In some cases, the system further comprises instructions that cause the processor to control the speed range of the polynucleotide in the first direction, second direction, or both. In some cases, the system further comprises instructions that cause the processor to control the voltage range of the first and second pores when the polynucleotide moves through the first and second pore in the first direction, second direction, or both.

In some cases, the system further comprises instructions that cause the processor to determine an optimal speed range of the polynucleotide in the first direction, second direction, or both, wherein the optimal speed range of the polynucleotide reduces the effect of Brownian motion on the polynucleotide. In some cases, adjusting voltages to create multiple scans at multiple different speeds improves the comprehensiveness of the data to which to map features. For example, at high speeds (i.e. when the voltage differential is larger), the molecules (e.g., polynucleotide, payload molecule, etc.) is more likely to be deterministic and the molecule is less affected by Brownian motion (e.g. Brownian motion will “pollute” the scanning data less). In some cases, the system determines the optimal speed at which one or more features can be detected before the molecule escapes the device or reverses direction. In some cases, the system further comprises instructions that cause the processor to determine the maximal speed at which Brownian motion least effects the molecule (e.g. maximal speed where Brownian motion is reduced). In some cases, the one or more features are charged so that they perturb the force and therefore the motion when the polynucleotide passes through the pores.

In some cases, controlling the speed range of the polynucleotide comprises determining the optimal speed range of the polynucleotide for sequencing.

In some cases, the system further comprises instructions that cause the processor to combine the speed of all the features and compute the time history of the speed of the polynucleotide in a given scan and given direction of scanning.

Aspects of the present disclosure include a dual-pore, dual-amplifier device for sequencing a polynucleotide sequence of a target polynucleotide through a first and a second pore, the device comprising: (i) an electrode connected configured to provide a first voltage at the first pore of the device, and provide a second voltage at the second pore of the device; (ii) the first pore; (iii) the second pore; wherein the first pore and the second pore are configured such that the target polynucleotide is capable of simultaneously moving across both pores in a first direction or a second direction, and in a controlled manner; (iv) one or more sensors capable of identifying: a barcode sequence, in a first cycle, from the target polynucleotide, during movement of the target polynucleotide through the first pore and the second pore in the first direction and, a second set of primers, in the first cycle, from the target polynucleotide, during movement of the target polynucleotide through the second pore and the first pore in the second direction; (v) a processor; and (vi) a non-transitory computer-readable medium comprising instructions that cause the processor to: a) determine, from the one or more sensors, the presence of the target polynucleotide in one or both pores; b) scan for one or more barcode sequences associated with the target polynucleotide; c) detect, in a first cycle, the barcode sequence(s) when the target polynucleotide traverses one or both pores in a first direction; d) when the barcode sequence(s) are detected in the first direction, adjust the first voltage, the second voltage, or both, to the first and/or second pore to change the direction of the target polynucleotide so that at least a portion of the target polynucleotide moves from the second pore to the first pore in a second direction; e) identify each nucleotide of the target polynucleotide that passes through one of the pores, by measuring an ionic current across the pore when the nucleotide passes that pore; f) direct the target polynucleotide into a second portion of the device (e.g., into a second channel coupled to the second pore, into a chamber coupled to at least one of the first pore and the second pore, etc.); g) direct non-target material, where the non-target material omits the one or more barcode sequences, into a third portion of the device (e.g., into a chamber coupled to at least one of the first pore and the second pore, etc.); h) removing the non-target material from the device; i) repeating steps a) through i) to enrich one or more target polynucleotides from the sample; and j) processing the target polynucleotide(s) (e.g., with amplification within the device, with amplification outside of the device, with delivery of the target polynucleotides from the device for downstream processing, etc.).

### Alternative Example Nanopore Device

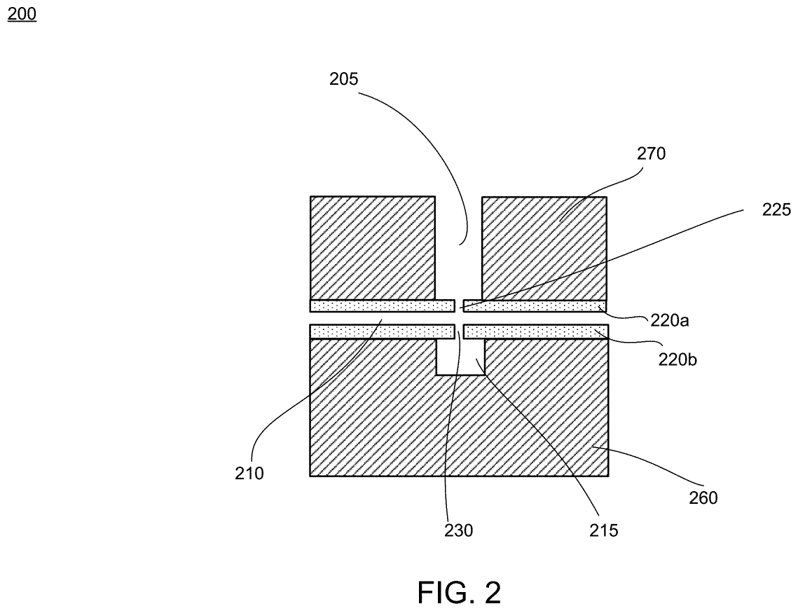

FIG. 2 depicts an additional example of a nanopore device 200 including a first nanopore 225 and a second nanopore 230, with chambers 205, 210, and 215. The depiction of the first chamber 205, second chamber 210, and third chamber 215 in FIG. 2 is shown as one example and does not indicate that, for instance, the first chamber is placed above the second or third chamber, or vice versa. The two nanopores 225 and 230 can be arranged in any position so long as they allow fluid communication between the chambers. Still, in one aspect, the nanopores are aligned as illustrated in FIG. 2.

In various embodiments, the alternative example nanopore device 200 shown in FIG. 2 for employing a two-nanopore, one-sensor configuration is a two chamber, two pore device. As an example, a two chamber, two pore device can include a first chamber and second chamber that are each in fluid communication with a first 225 and second nanopore 230, respectively. A plurality of layers can separate the two chambers. For example, the plurality of layers comprise: a first layer 260; a second layer 270; and a conductive middle layer 220a, 220b disposed between the first and second layers. In this two chamber, two pore device, the first nanopore 225 and second nanopore 230 may be connected to one another through a channel that is located within the conductive middle layer. A channel refers to any fluid path that enables fluid flow between the first nanopore 225 and second nanopore 230.

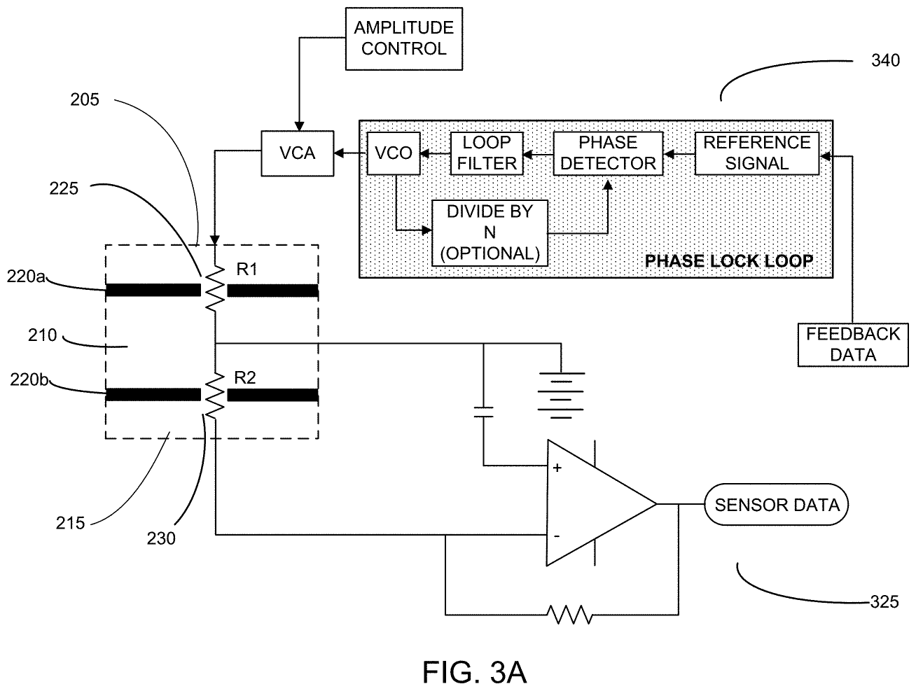

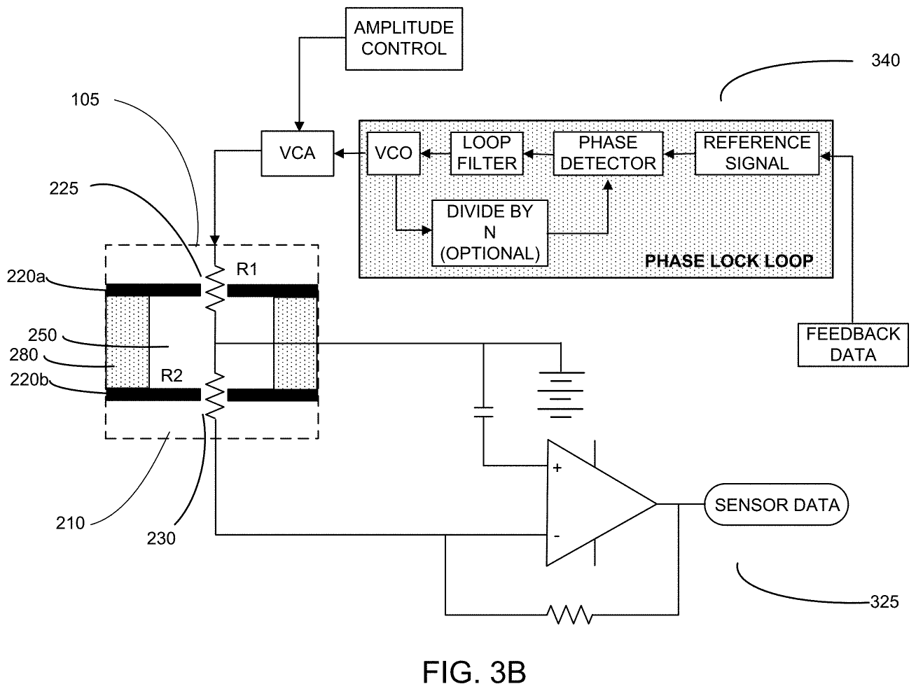

FIGS. 3A-3B depict example circuitry incorporating the first 225 and second nanopores 230 of an example nanopore device, in accordance with two embodiments, as described in applications incorporated by reference above. As shown in FIG. 3A, sensing and controlling of a molecule can occur while at least a portion of the molecule resides within the second chamber 210. Additionally, FIG. 3B depicts a configuration in which sensing and controlling of a molecule can occur while at least a portion of the molecule resides within the channel 250. Although the embodiments depicted in FIGS. 3A and 3B depict two nanopores, the circuitry design can be applied to more than two nanopores, where sensing and controlling a molecule can be performed at any of the multiple nanopores.

### Switchable Sensing and Control Circuitry

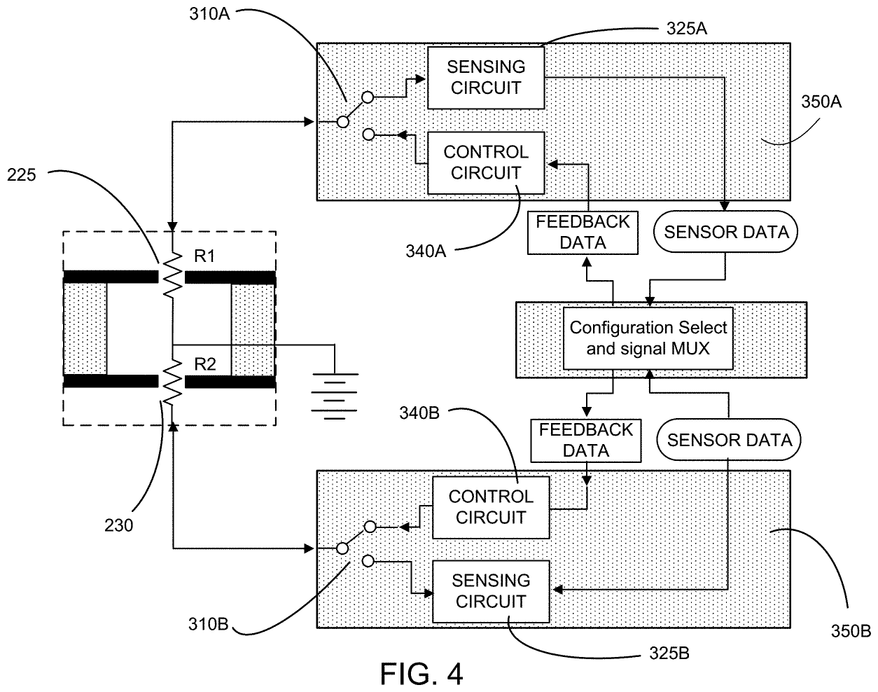

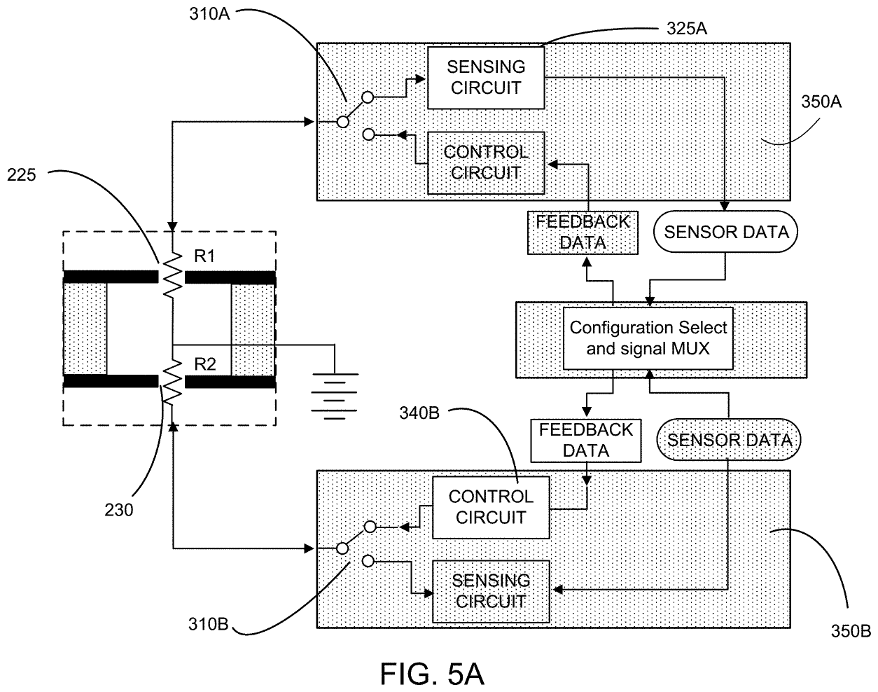

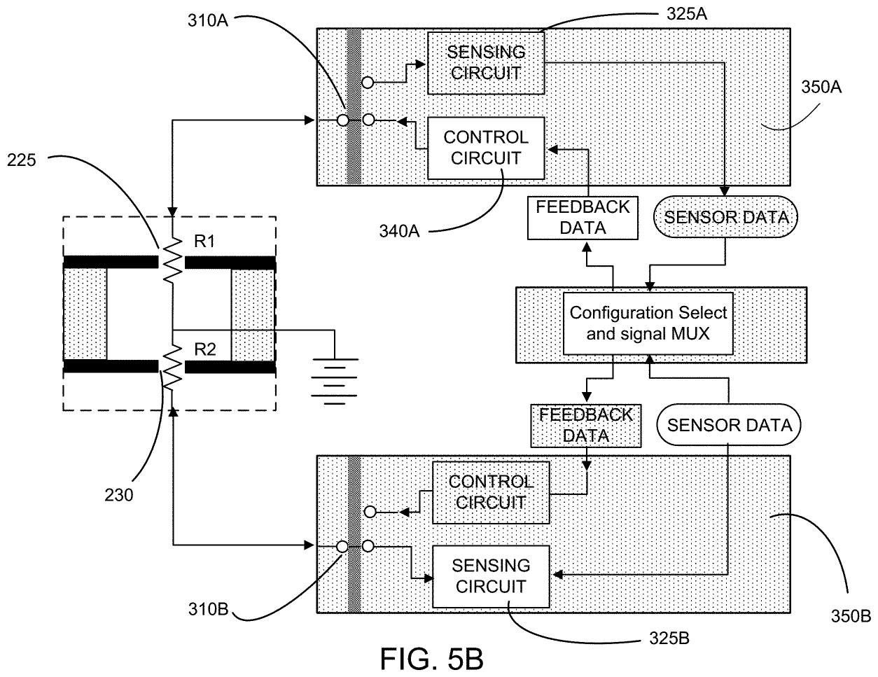

In various embodiments, the sensor and control circuitry options are available at each of the two pores. FIG. 4 depicts an example two nanopore device with a sensing circuitry 325 and a control circuitry 340 option for each nanopore, and a switch 310 between the two options for each pore, in accordance with one embodiment. In particular, a first nanopore 225 is incorporated in a first overall circuitry 350A that includes a first set of both a sensing circuitry 325A and a control circuitry 340A. Additionally, a second nanopore 230 is incorporated in a second overall circuitry 350B that includes a second set of both a sensing circuitry 325B and a control circuitry 340B. Each overall circuitry 350 includes a switch 310A and 310B that enables switching between a sensing circuitry 325 and control circuitry 340 of each overall circuitry 350. In one embodiment, setting each switch 310 can enable sensing across the first nanopore 225 and control at a second nanopore 230, or vice versa. In various embodiments, the switches 310A and 310B may be embodied differently than displayed in FIG. 3. For example, certain hardware components may be shared between the sensing circuitry 225 and control circuitry 240 and therefore, each switch 310 can be configured such that the function of each circuitry (including the requisite hardware components) is appropriately enabled when desired (e.g., as in FIG. 5A and FIG. 5B). The embodiments are further described in applications incorporated by reference above.

### Operation of Multi-Pore Devices

Generally, a control circuitry 240 and a sensor circuitry 225, as shown in FIGS. 3A and 3B, or multiple control circuitries 340A, 340B and multiple sensor circuitries 325A, 325B, as shown in FIGS. 4 and 5A-5B can be employed together in a two pore one sensor device to control the movement of a molecule (e.g., polymer, polynucleotide, vector, protein, etc.), for sensing and data collection. Although the subsequent description refers to the two nanopore device in a second configuration state (e.g., sensing circuitry 325B incorporating the second nanopore 230 and control circuitry 340A incorporating the first nanopore 225), the description can similarly be applied to additional configuration states (e.g., first configuration state).

For example, in the two pore device depicted in FIGS. 3A and 3B, the control circuitry 340 applies a dynamically altered voltage across the first nanopore 225 that generates a force that directionally opposes the force generated by the static voltage applied across second nanopore 230 by the sensor circuitry 325, with a dynamic magnitude that results in controlled motion of the molecule in either direction. In particular, the voltage applied by the control circuitry 340 across the first nanopore 225 can direct the movement of molecules by generating varying field force strengths that are in magnitude larger than, equal to, or less than the static force deriving from the voltage applied to the second nanopore 230 by the sensor circuitry 325. Therefore, dynamic adjustment of the voltage field force at the first nanopore 225, relative to the static field force at the second nanopore 330, enables control over the net direction of motion of a molecule as well as the rate of motion (e.g., velocity) of a molecule situated between both nanopores 225 and 230 in either the middle chamber 210 or channel 250.

In a related example, in the two pore device depicted in FIGS. 4A and 4B, the control circuitry 340 applies a driving force using an AC electric field with an associated AC frequency. Control or selection of the AC frequency (or another aspect of the AC electric field applying the driving force) can be based upon information from the sensor circuitry 325. For instance, one or more of frequency (e.g., frequency at which a target passes back and forth through a nanopore), amplitude of a signal, phase of a signal, event duration (e.g., associated with target motion at a pore), quantity of targets, and/or any other suitable feature of an electrical signal from the sensor circuitry 325 can be used to dynamically adjust aspects of the AC electric field applying the driving force of the control circuitry 340. Therefore, a driving force from an AC source at one nanopore (e.g., the second nanopore 230) can enable control over the net direction of motion of a molecule as well as the rate of motion (e.g., velocity) of a molecule situated between nanopores 225, 230.

In particular, the dynamic voltage applied by the control circuitry 340 can have a phase that is shifted in comparison to the phase of the sensor data gathered by the sensor circuitry 325. Therefore, as the molecule passes through the second nanopore 230 in a first direction, the applied dynamic voltage changes such that the force imparted by the dynamic voltage opposes the direction of movement of the molecule. The molecule then changes directions and passes through the second nanopore 230 in a second direction (e.g., opposite of the first direction). Here, the dynamic voltage changes again to oppose the second direction of movement of the molecule. This process can be repeated to enable the molecule to pass back and forth through the second nanopore 230 until a sufficient measurement of the segment of the molecule is obtained.

By oscillating the less-than or greater-than force at the first nanopore 225, relative to the static force at the second nanopore 230, the segments of the molecule can be sensed many times by the sensor circuitry 325B by repeatedly passing the molecule through the second nanopore 230. Doing so can improve the signal of detected ionic changes corresponding to translocation of the molecule across the second nanopore 230 which is useful for a variety of signal processing purposes, e.g., to improve sequencing of a molecule such as DNA. The repeated back and forth passing of the molecule, such as a polynucleotide, through the second nanopore 230 is referred to as “flossing” of the polynucleotide. Specifically, the flossing of the DNA segment (or a portion of the DNA segment) through the second nanopore 230 is in response to applied forces (e.g., electrical forces derived from the applied voltages) and can further include frequency data corresponding to the rate of translocation of the DNA segment through the second nanopore 230. As an example, the frequency data is the period of a single nucleotide base that begins at an initial position, translocates across the second nanopore 230 in a first direction (e.g., enter into middle chamber 210 or leave middle chamber 210), translocates back across the second nanopore 230 in a direction opposite to the first direction, and returns to the initial position.

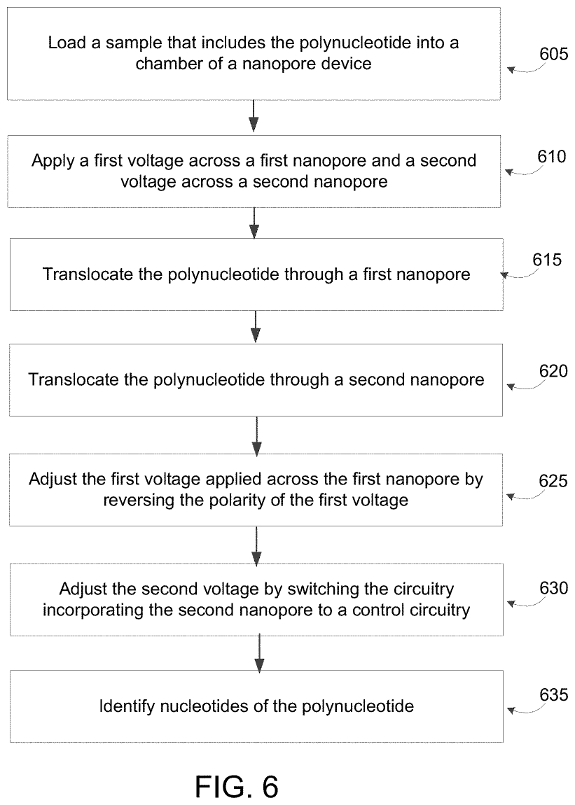

FIG. 6 depicts a flow process for sequencing a molecule such as a polynucleotide, in accordance with an embodiment. Specifically, a sample that includes the polynucleotide is loaded 605 into a first chamber of a nanopore device. In some embodiments, the polynucleotide can be loaded into a different chamber (e.g., third chamber 215 as shown in FIG. 3A or second chamber 210 in FIG. 3B). The two nanopore device applies 610 a first voltage across a first nanopore and a second voltage across a second nanopore. In various embodiments, this can be accomplished by placing the two nanopore device in a third configuration state (e.g., both the first nanopore and second nanopore are incorporated in sensing circuitries). Therefore, the first and second voltages are each applied by a sensing circuitry. The polynucleotide translocates 615 from the first chamber and through a first nanopore. Specifically, the sensor circuitry of the first nanopore can apply a constant voltage across the first nanopore that generates an electrical force that draws the polynucleotide through the first nanopore. The sensor circuitry may be configured to measure changes in ionic current through the first nanopore. Therefore, when the polynucleotide translocates through the first nanopore, the sensor circuitry detects the translocation event based on a detected change in ionic current. Additionally, the polynucleotide translocates 620 through the second nanopore due to the applied voltage by the sensor circuitry.

The two nanopore device may switch into a different configuration that opposes the direction of the movement of the molecule. For example, the two nanopore device switches from a third configuration state to a first configuration state or a second configuration state depending on the directional movement of the molecule. If the molecule was initially loaded into the first chamber, then the molecule is directionally exiting from the first chamber and moving towards the second or third chamber. Therefore, to oppose the movement of the molecule, the two nanopore device can switch from a third configuration into a first configuration state (e.g., see FIG. 5A). In some embodiments, if the molecule was initially loaded into a third chamber or second chamber, then the molecule is directionally moving towards the first chamber 105. Therefore, to oppose the movement of the molecule, the two nanopore device can switch from a third configuration into a second configuration state (e.g., see FIG. 5B).

The subsequent description refers to switching the two nanopore device to a first configuration state, but can also be applied for a switch to the second configuration state. In various embodiments, the first voltage applied by the circuitry incorporating the first nanopore is adjusted 625. Specifically, the polarity of the sensing circuitry is set such that it opposes the movement of the molecule. For example, the polarity of sensing circuitry can be reversed from a first polarity in the third configuration state to a reverse of the first polarity in the first configuration state. Additionally, the second voltage applied by the circuitry incorporating the second nanopore is also adjusted 630. Specifically, the control circuitry of the second overall circuitry applies an adjusted second voltage across the second nanopore in response to detecting that the polynucleotide has translocated through the first nanopore. Generally, the magnitude of the adjusted second voltage applied by the control circuitry is dynamically changing (e.g., an oscillating voltage) such that the electrical force arising due to the adjusted second voltage can oppose the static force arising from the adjusted first voltage. The second voltage applied by the control circuitry 240 has a particular waveform (e.g., varying amplitude/magnitude at a particular frequency) such that the polynucleotide can similarly oscillate back and forth through the first nanopore. As the polynucleotide oscillates, the sensor circuitry can detect ionic current changes through the first nanopore that corresponds to the translocation of nucleotide bases of the polynucleotide. Each nucleotide base can be read multiple times as the polynucleotide flosses back and forth through the first nanopore, thereby enabling the more accurate identification 635 of individual nucleotides of the polynucleotide.

When a single nucleotide base from the polynucleotide has been sufficiently read, a polynucleotide exit state in the applied second voltage can be applied by the control circuitry to allow for DNA segment incrementation. In other words, the second voltage can be temporarily adjusted to allow a subsequent nucleotide base pair to translocate through the first nanopore, at which point the second voltage can be resumed to floss the subsequent nucleotide base pair back and forth through the first nanopore. The magnitude and frequency of the applied second voltage across the second nanopore by the control circuitry can be tailored according to frequency information corresponding to the ionic current measurements detected by the sensor circuitry.

In various embodiments, an automated and functional circuitry (e.g., using state machine or machine learning algorithms in concert with feedback control) could control both the sensor circuitry and the control circuitry, to continuously monitor the sensed data. Therefore, a section of DNA can be read for optimal performance. For example, if the ion current corresponding to a DNA translocation event through the first nanopore is not resolved, then the control circuitry can perform a step-wise increase in the applied voltage across the second nanopore. Doing so increases the force opposing the static force applied by the sensor circuitry, thereby slowing the movement of a DNA segment as it translocates through the first nanopore. This improves the signal to noise ratio for each DNA translocation across the first nanopore until the desired performance (e.g., signal resolution) is achieved.

Passing a polynucleotide segment and sensing the segment multiple times using a sensing circuitry enables the reduction of signal error to an acceptable level. Alignment of signals can be used to achieve consensus sequences with acceptable accuracy. In some embodiments, the multiple reads corresponding to multiple DNA translocations can be used to generate a consensus signal, which can subsequently be used to identify the nucleotide base pair.

Sequencing and/or feature detection can additionally or alternatively be performed as described in applications incorporated by reference above.

### Material Sorting and Other Applications

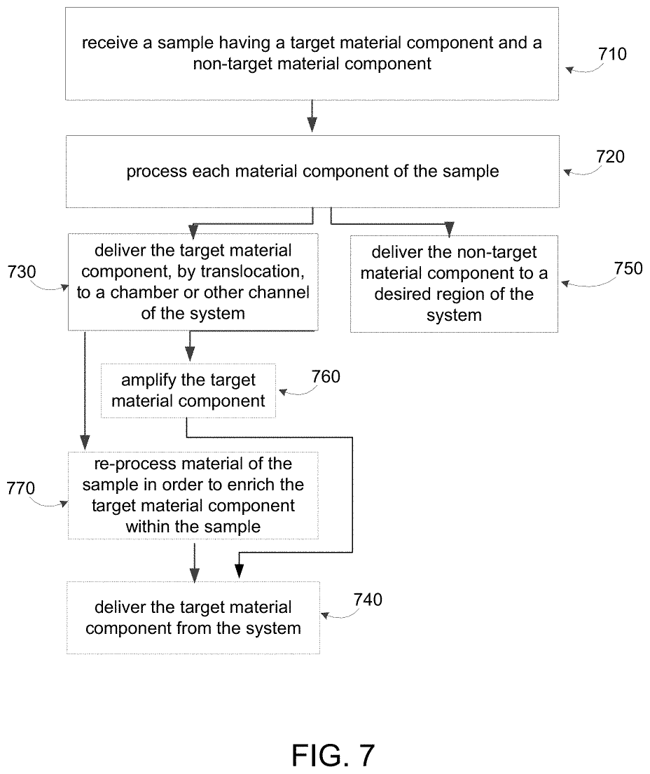

In some applications, system component(s) described can implement methods for sorting material in a manner that allows for selective retrieval of target material from a sample, discrimination of target material from non-target material of a sample, and/or enrichment of target material within a sample. In embodiments of a method 700, as shown in FIG. 7 the system(s) can thus: receive 710 a sample having a target material component (e.g., target molecules) and a non-target material component (e.g. non-target molecules); process 720 each material component of the sample (as described above) using control and sensing circuitry of the system; deliver 730 the target material component, by translocation, to a chamber or other channel of the system (e.g., region 105, 110, 115, 125, or 130 of the system); and deliver the target material component 740 from the system for downstream processing or other applications. In some variations, the system can perform one or more of: delivering 750 the non-target material component to a desired region of the system (e.g., for retrieval or discarding); amplifying 760 the target material component within the device and/or away from the device; re-processing 770 material of the sample in order to enrich the target material component within the sample; and perform other suitable operations.

In embodiments, the system(s) and methods discussed enable enrichment of target amplicons from background (e.g., for cell-free DNA analysis), with a single-molecule approach. The approach provides systems and methods for serially detecting and then fluidically sorting molecules, to segregate target molecules from non-target molecules, that can work upstream of PCR or non-PCR workflows. Discussed methods can also segregate other types of target analytes, including chromosomal fragments comprising histones that are detected as having a target modification, from those fragments with histones that do not have the modification, and sorting facilitating enriching for the modified histone containing chromosomal fragment for subsequent epigenetic analysis, such as ChIP-seq or ATAC-seq or bisulfate sequencing.

The method 700 can be implemented by embodiments, variations, and examples of the nanopore devices described above.

In more detail, a nanopore device can receive 710 a sample having a target material component (e.g., target molecules) and a non-target material component (e.g. non-target molecules), such as into one of ports 126, 127, 131, and 132 of the nanopore device 100 or chamber 110 of the nanopore device shown in FIG. 1 (or other channels of nanopore devices, as described above). As described above, the sample can be a biological sample having a population of target molecules (e.g., polymers, polynucleotides, viral vectors, plasmids, proteins, etc.) and non-target material, whereby the system receives 710 the sample and its components into a channel (e.g., first channel 125 or second channel 130 shown in FIG. 1) of the nanopore device for characterization and processing in subsequent steps.

The nanopore device can then process 720 each material component of the sample (as described above) using control and sensing circuitry of the nanopore device. In variations, the nanopore device can translocate a polynucleotide of the sample from a first location within the nanopore device, into a nanopore (e.g., first nanopore 105 shown in FIG. 1, second nanopore 115 shown in FIG. 2) coupled to a channel (e.g., first channel 125, second channel 130, etc.) of the nanopore device, upon application of a control voltage across the first nanopore by a control circuit of the nanopore. Upon translocation of the polynucleotide into one or more nanopores of the nanopore device, the system can detect features of the polynucleotide through sequencing or through other means described above, in order to determine whether the polynucleotide is a target material component or a non-target material component.

In variations, the nanopore device can generate signals from processing material in order to detect features of target material and non-target material used for sorting. In particular, generating signals can include translocating the polynucleotide into a nanopore (e.g., the first nanopore, the second nanopore, etc.) and applying a sensing voltage across the nanopore by a sensing circuit of the nanopore. Features used for discrimination of target material from non-target material can include one or more of: sequence length (e.g., long-read sequences, short-read sequences, etc.) based on determination of area under the curve of signal vs. time, barcodes associated with target material (e.g., through pre-processing the sample to tag target material with barcode sequences), tagging with detectable markers, physical features (e.g., of plasmids, of viral vectors) of target material and non-target material, other structures (e.g., of nucleic acid origami libraries), other features of single or double stranded polynucleotides, or other suitable features. Individual features and combinations of features can then be used as detectable signatures to determine if a processed component of the sample is a target component or a non-target component.

After processing the target and non-target components of the sample, the nanopore device can then deliver 730 the target material component, by translocation, to a chamber or other channel of the system (e.g., region 105, 110, 115, 125, or 130 of the system shown in FIG. 1). In particular, the system can control voltages associated with different environments of the nanopore device, in order to direct detected target material to a first location and to direct non-target material to a second location.

In variations of step 730, the nanopore device can translocate each target polynucleotide detected from the sample, from an initial location into the first channel 125 by way of the first nanopore 105, into the second channel 130 by way of the second nanopore 115, or into the common chamber 110. Similarly, n variations of step 730, the nanopore device can translocate each non-target polynucleotide detected from the sample, from an initial location into the first channel 125 by way of the first nanopore 105, into the second channel 130 by way of the second nanopore 115, or into the common chamber 110. As such, an initial mixed sample can be sorted into different regions (e.g. the first channel 125, the second channel 130, the chamber 110) of the nanopore device.

After sorting, the deliver the target material component 740 from the system for downstream processing or other applications. In variations, all sorted target molecules can be delivered from the first channel 125 (e.g., through ports 126, 127 shown in FIG. 1), from the second channel 130 (e.g., through ports 131, 132 shown in FIG. 1), or from the common chamber 110 shown in FIG. 1. Delivery can be performed through application of positive pressure to volumes of the nanopore device and/or through negative pressure. For instance, the system can include a pressurized heading or other pumping system to pull or push the target material component from the nanopore device for additional processing. Additionally or alternatively, channels of the nanopore device can be asymmetric in design (e.g., in relation to channel cross section, in relation to volume, in relation to other channel morphology, etc.) in order to facilitate delivery of the target material component from the nanopore device.

In some variations, the system can additionally deliver 750 the non-target material component to a desired region of the system (e.g., for retrieval or discarding). In variations, all sorted non-target molecules can be delivered from the first channel 125 (e.g., through ports 126, 127 shown in FIG. 1), from the second channel 130 (e.g., through ports 131, 132 shown in FIG. 1), or from the common chamber 110 shown in FIG. 1. Delivery can be performed through application of positive pressure to volumes of the nanopore device and/or through negative pressure. For instance, the system can include a pressurized heading or other pumping system to pull or push the non-target material component from the nanopore device for additional processing. Additionally or alternatively, channels of the nanopore device can be asymmetric in design (e.g., in relation to channel cross section, in relation to volume, in relation to other channel morphology, etc.) in order to facilitate delivery of the non-target material component from the nanopore device.

In some variations, the system can additionally perform amplification of 760 the target material component within the nanopore device and/or away from the nanopore device. In variations where the target material component is delivered from the nanopore device, other system elements (e.g., thermocycling subsystems, fluid handling subsystems, etc.) can perform amplification (e.g., with respect to polymerase chain reaction operations) away from the nanopore device in order to amplify the target content prior to additional processing and characterization.

Additionally or alternatively in some variations, the system can retain the target material component within a region of the nanopore device (e.g., chamber 110, channel 125, or channel 130, other region of the nanopore device shown in FIG. 1, other region of nanopore devices described) in order to perform an on-device reaction or other process. For instance, in relation to amplification (e.g., polymerase chain reaction, PCR), the system can perform on-device amplification of target material using a PCR apparatus (described below, and for instance, due to thermal and optical characteristics of the chambers of the system) or other PCR apparatus. The system can then deliver amplified target material from the system for retrieval and/or performance of downstream analyses or other processes, as described in relation to step 740 above.

In some variations, the system can re-process 770 material of the sample in order to enrich the target material component within the sample. For instance, after removal of non-target material from the nanopore device (e.g., with flushing of non-target material from chamber 110 shown in FIG. 1) subsequent to a first sorting run of the system, the nanopore device can then re-process the remainder of the sample by sensing signals indicative of target material and non-target material as described in relation to step 720 above, and further sort any remaining non-target material from target material based upon the signals and feature extraction to discriminate target molecules based upon identified signatures. Re-processing can include reversing applied voltages or otherwise adjusting electrical parameters of the nanopore device in order to reverse motion of the remaining material within the nanopore device, followed by re-scanning of the remaining material. Then, with further removal (e.g., flushing) of non-target material from the nanopore device, the target material constituent of a sample can be further enriched for downstream processing. Step 770 can be performed any number of times, in order to achieve a desired level of enrichment of target material from the sample.

According to applications of use of the sorted target molecules, the method 700 can further include steps for or support one or more of: amplification of long-read sequences; identification of genetic variants (e.g., of bacteria) associated with antibiotic resistance, based upon barcoding target regions of a polynucleotide; identification of genetic variants associated with drug resistance, based upon barcoding target regions of a polynucleotide; enrichment of bacteria from whole blood based upon sorting of bacteria from a blood sample; capture of plasmids; sorting of wild-type and non-wild-type genetic variants; sorting of lentiviral vectors from a sample; identification and sorting of proteins (e.g., IgM antibodies, IgD antibodies, IgG antibodies, IgA antibodies, IgE antibodies, other proteins, etc.); sorting of whole phages (e.g., 20-200 nm phages); generation of aptamer libraries; screening of nucleic acid origami libraries to find new structures; identification and sorting of molecules that can be used as barcoding agents; segregation of chromosomal fragments comprising histones that are detected as having a target modification, from those fragments with histones that do not have the modification; sorting facilitating enriching for the modified histone containing chromosomal fragment for subsequent epigenetic analysis, such as ChIP-seq or ATAC-seq or bisulfite sequencing; and performing other suitable applications.

In one embodiment, a method implemented by an embodiment, variation, or example of the system can include: receiving a sample, comprising the polynucleotide, at a first channel of a nanopore device; translocating the polynucleotide into a first nanopore coupled to the first channel, upon application of a control voltage across the first nanopore by a control circuit of the first nanopore; generating a signal upon translocating the polynucleotide into the first nanopore and applying a sensing voltage across the first nanopore by a sensing circuit of the first nanopore; detecting a signature of the polynucleotide from the signal; and based upon the signature, translocating the polynucleotide into a second nanopore coupled to a second channel of the nanopore device. In embodiments, the sensing voltage is a constant voltage and wherein the control voltage is a dynamic voltage governing motion of the polynucleotide between the first channel and the second channel of the nanopore device. In embodiments, the signature of the polynucleotide is representative of one or more of: a length of the polynucleotide, a sequence of a region of the polynucleotide, and a structure of the polynucleotide. In embodiments, the method can further include: categorizing the polynucleotide as a target polynucleotide upon analyzing the signature, and retaining the polynucleotide within the second channel. In embodiments, the method can further include: transmitting heat toward the polynucleotide, and amplifying the polynucleotide within the nanopore device. In embodiments, the method can further include: categorizing the polynucleotide as non-target material upon analyzing the signature, and translocating the polynucleotide into the second channel or another chamber as non-target material waste. In embodiments, the method can further include: repeatedly reversing a polarity of the control voltage in response to detection of the signature, thereby repeatedly reversing motion of the polynucleotide across the first nanopore, and generating a subsequent set of signals from the polynucleotide. In embodiments, the method can further include: performing a validation operation with the signal and the subsequent set of signals, the validation operation configured to verify an identity of the polynucleotide from a confidence value determined from the signal and the subsequent set of signals. In embodiments, the method can further include: identifying features associated with the signature, wherein identifying features comprises: for an initial oscillation of the control voltage, detecting a first change in ionic current across the first nanopore corresponding to motion of a first region of the polynucleotide; and for a subsequent oscillation of the control voltage, detecting a second change in ionic current across the first nanopore corresponding to motion of a second region of the polynucleotide.

In one embodiment, a method implemented by an embodiment, variation, or example of the system can include: receiving the sample into a first channel of a nanopore device; translocating each of the subset of target material and the subset of non-target material into a first nanopore coupled to the first channel, upon application of a first voltage across the first nanopore by a control circuit of the first nanopore; generating a set of signals upon application of a sensing voltage across the first nanopore by a sensing circuit of the first nanopore; detecting, from the set of signals, a first subset of signatures characteristic of the subset of target material and a second subset of signatures characteristic of the subset of non-target material; translocating the subset of target material into a second channel of the nanopore device in response to detection of the first subset of signatures; and transmitting the subset of non-target material into a discard region of the nanopore device in response to detection of the second subset of signatures. In embodiments, the first subset of signatures and the second subset of signatures are associated with one or more of: a range in polynucleotide length, a polynucleotide sequence, and a polynucleotide structure. In embodiments, the sensing voltage is a constant voltage and wherein the control voltage is a dynamic voltage. In embodiments, the method can further include: dynamically adjusting the control voltage, thereby translocating at least one of the subset of target material and the subset of non-target material repeatedly in a forward direction and a reverse direction across the first nanopore. In embodiments, the method can further include: transmitting heat toward the second channel of the nanopore device and amplifying polynucleotides of the set of target material within the nanopore device. In embodiments, transmitting the subset of non-target material into the discard region comprises dynamically adjusting the control voltage, for each instance of detection of the second subset of signatures, thereby diverting the subset of non-target material into the discard region of the nanopore device. In embodiments, the method can further include delivering the subset of target material from the second channel of the nanopore device for further processing.

In embodiments, a system for sorting material of a sample comprising a subset of target material and a subset of non-target material can include: a first channel coupled to a first nanopore, and a second channel coupled to a second nanopore, the first nanopore and the second nanopore coupled to a common chamber (e.g., as described above); and a processor comprising a non-transitory computer-readable medium comprising instructions stored thereon, that when executed by the processor perform steps of one or more methods described above.

### Additional Considerations

While embodiments, variations, and examples of two pore devices and methods implemented with two pore devices are described above, alternative embodiments, variations, and examples of the invention(s) described can involve a non-two pore device. For instance, in variations, second chamber 110 (and variations described thereof) can be a conductive channel of a single pore device, wherein the single pore device has control circuitry (e.g., by way of gate voltage), sensing circuitry (e.g., in relation to source-to-drain current flow), with the ability to switch between control circuitry and sensing circuitry. Such a single pore device can be manufactured with a lithography process, a drilling process, or any other suitable process that generates a channel or chamber through layers of material.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A method for processing a sample comprising a subset of target polynucleotides and a subset of non-target polynucleotides, wherein processing comprises one or more of sorting and characterizing the sample, the method comprising:
receiving a target polynucleotide of the subset of target polynucleotides, at a first channel of a nanopore device;
translocating the target polynucleotide into a first nanopore coupled to the first channel, upon application of a control voltage across the first nanopore by a control circuit of the first nanopore;
generating a target signal from the target polynucleotide upon translocating the target polynucleotide into the first nanopore and applying a sensing voltage across the first nanopore by a sensing circuit of the first nanopore;
detecting a signature characteristic of the target polynucleotide from the target signal; and
based upon the signature, translocating the target polynucleotide into a second region of the nanopore device.

2. The method of claim 1, further comprising:
receiving a non-target polynucleotide of the subset of non-target polynucleotides, at the first channel of a nanopore device;
translocating the non-target polynucleotide into the first nanopore coupled to the first channel, upon application of a control voltage across the first nanopore by a control circuit of the first nanopore;
generating a non-target signal from the non-target polynucleotide upon translocating the non-target polynucleotide into the first nanopore and applying the sensing voltage across the first nanopore by the sensing circuit of the first nanopore; and
based upon the non-target signal, translocating the non-target polynucleotide into a discard region of the nanopore device.

3. The method of claim 2, wherein the sensing voltage is a constant voltage and wherein the control voltage is a dynamic voltage governing motion of the polynucleotide between the first channel and the second channel of the nanopore device.

4. The method of claim 1, wherein the second region of the nanopore device comprises one of a) a second channel coupled to a second nanopore of the nanopore device and b) a common chamber in fluid communication with the first channel and the second channel.

5. The method of claim 4, wherein the second nanopore is positioned less than or equal to 5 micrometers from the first nanopore.

6. The method of claim 2, wherein the discard region of the nanopore device comprises one of a) a second channel coupled to a second nanopore of the nanopore device and b) a common chamber in fluid communication with the first channel and the second channel.

7. The method of claim 6, further comprising flushing the non-target polynucleotide from the third portion of the nanopore device.

8. The method of claim 1, wherein the signature of the target polynucleotide is representative of one or more of: a length of the polynucleotide, a sequence of a region of the polynucleotide, and a structure of the polynucleotide.

9. The method of claim 1, further comprising labeling the target polynucleotide with a barcode sequence, and wherein the signature of the target polynucleotide is representative of the barcode sequence.

10. The method of claim 1, further comprising reversing a polarity of the control voltage in response to detection of the signature of the target polynucleotide, thereby repeatedly reversing motion of the polynucleotide across the first nanopore and re-sorting the target polynucleotide.

11. The method of claim 1, further comprising identifying features of the target polynucleotide associated with the signature, wherein identifying features comprises:
for an initial oscillation of the control voltage, detecting a first change in ionic current across the first nanopore corresponding to motion of a first region of the target polynucleotide; and
for a subsequent oscillation of the control voltage, detecting a second change in ionic current across the first nanopore corresponding to motion of a second region of the target polynucleotide.

12. The method of claim 1, further comprising amplifying the target polynucleotide within the nanopore device with transmission of heat toward the nanopore device.

13. The method of claim 1, wherein material comprising the target polynucleotide comprises a polynucleotide-protein complex.

14. The method of claim 1, wherein the subset of target polynucleotides comprises genetic material associated with antibiotic resistance, the method comprising generating a characterization of antibiotic resistance within the sample.

15. The method of claim 1, wherein the subset of target polynucleotides comprises genetic material associated with drug resistance, the method comprising generating a characterization of drug resistance within the sample.

16. The method of claim 1, wherein the subset of target polynucleotides comprises one of wild-type genetic material and non-wild-type genetic material, the method comprising generating a characterization of wild-type composition of the sample.

17. The method of claim 1, wherein the subset of target polynucleotides comprises a viral polynucleotide.

18. The method of claim 1, wherein the subset of target polynucleotides comprises a bacterial polynucleotide, and wherein the sample comprises whole blood.

19. A method for sorting material of a sample comprising a subset of target material and a subset of non-target material, the method comprising:
receiving the sample into a first channel of a nanopore device;
translocating each of the subset of target material and the subset of non-target material into a first nanopore coupled to the first channel, upon application of a first voltage across the first nanopore by a control circuit of the first nanopore;
generating a set of signals upon application of a sensing voltage across the first nanopore by a sensing circuit of the first nanopore;
detecting, from the set of signals, a first subset of signatures characteristic of the subset of target material and a second subset of signatures characteristic of the subset of non-target material;
translocating the subset of target material into a second region of the nanopore device in response to detection of the first subset of signatures; and
transmitting the subset of non-target material into a discard region of the nanopore device in response to detection of the second subset of signatures.

20. The method of claim 19, wherein the first subset of signatures and the second subset of signatures are associated with one or more of: a barcode sequence, a range in polynucleotide length, a polynucleotide sequence, and a polynucleotide structure.

21. A system for sorting material of a sample comprising a subset of target material and a subset of non-target material, the system comprising:
a first channel, a second channel, and a common chamber;
a first nanopore providing communication between the first channel and the common chamber, wherein the first nanopore comprises a first sensing circuit and a first control circuit;
a second channel providing fluid communication between the common chamber and the second channel; and
a processor comprising a non-transitory computer-readable medium comprising instructions stored thereon, that when executed by the processor perform the steps of:
translocating each of the subset of target material and the subset of non-target material into the first nanopore, upon application of a first voltage across the first nanopore by the first control circuit,
generating a set of signals upon application of a sensing voltage across the first nanopore by the sensing circuit;
detecting, from the set of signals, a first subset of signatures characteristic of the subset of target material and a second subset of signatures characteristic of the subset of non-target material;
translocating the subset of target material into a second channel of the nanopore device in response to detection of the first subset of signatures; and
transmitting the subset of non-target material into a discard region of the nanopore device in response to detection of the second subset of signatures.

22. The system of claim 21, further comprising a heating element configured to transmit heat toward a portion of the nanopore device, the processor further comprising instructions for amplification of polynucleotides of the subset of target material within the nanopore device.

23. The system of claim 21, further comprising a voltage control subsystem in communication with at least one of the first nanopore and a second nanopore, wherein the first nanopore is positioned less than or equal to 5 micrometers from the second nanopore, and wherein the voltage control subsystem implementing a direct current-biased alternating current signal source.
